(19) United States
(12) Patent Application Publication
(10) Pub. No.: US 2005/0287574 A1
(43) Pub. Date: Dec. 29, 2005

(54) Genetic diagnostic method for SCD risk stratification

(75) Inventors: Orhan Soykan, Shoreview, MN (US); Daisy Phan Cross, Minneapolis, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Medtronic, Inc., Fridley, MN (US)

(21) Appl. No.: 11/157,532

(22) Filed: Jun. 21, 2005

Related U.S. Application Data
(60) Provisional application No. 60/589,907, filed on Jul. 21, 2004.
(60) Provisional application No. 60/582,352, filed on Jun. 23, 2004.

(51) Int. Cl. C12Q 1/68

(52) U.S. Cl. 435/6

(57) ABSTRACT

A method of classifying individuals as to their risk of experiencing sudden cardiac death (SCD) utilizes single nucleotide polymorphisms (SNPs). A genotype profile, which includes one or more SCD-associated SNPs, is generated for an individual. The probability of experiencing SCD is determined based on the genotype profile.

### CROSS-REFERENCE TO RELATED APPLICATIONS

This application claims the benefit of U.S. Provisional Application No. 60/589,907 filed on Jul. 21, 2004, for “Genetic Diagnostic Method for SCD Risk Stratification” by O. Soykan and D. Cross.

This application is related to applications entitled “Self-Improving Classification System” and “Self-Improving Identification Method,” which were filed on the same day and also assigned to Medtronic, Inc.

### BACKGROUND OF THE INVENTION

The present invention relates to kits and methods for classifying individuals based on their risk for sudden cardiac death (SCD). In particular, the diagnosis of risk is based on the presence of one or more single nucleotide polymorphisms (SNPs) associated with SCD.

Implantable medical devices (IMDs), such as defibrillators, effectively terminate life-threatening ventricular tachy-arrhythmias. Examples of ventricular tachy-arrhythmias include ventricular tachycardias and ventricular fibrillation. IMDs are indicated for many individuals with a variety of cardiac-related ailments such as myocardial infarction, ischemic heart disease, coronary artery disease, and heart failure. The use of IMDs, however, remains low, in part due to the lack of reliable predictors to select individuals who would truly benefit from the devices.

Mean sensitivity and specificity values for markers currently used for risk stratification of individuals for myocardial infarction are listed in Table 1.

TABLE 1

| | | | | Left | |
|---|---|---|---|---|---|
| | | Severe | | Ventricular | |
| | HR | Ventricular | Signal | Ejection | |
| | Variability | Arrhythmia | Averaged | Fraction | EP |
| Test | on AECG | on AECG | ECG | (EF) | Studies |
| Sensitivity | 49.8% | 42.8% | 62.4% | 59.1% | 61.8% |
| Specificity | 85.8% | 81.2% | 77.4% | 77.8% | 84.1% |

Although most of the specificity values are high, sensitivity values are low. The most commonly used marker, EF, has a sensitivity of about 59%. This means that about 41% of individuals are missed if EF is the only marker utilized. Although electrophysiology (EP) studies provide slightly better sensitivity and specificity results, they are rather invasive and, therefore, performed infrequently.

A single nucleotide polymorphism (SNP) is a small genetic variation that occurs throughout an individual's genome. Each individual's genome contains a unique SNP profile that is made up of many different genetic variations. Current studies have found that SNPs may cause disease or, more frequently, are markers that may diagnose disease.

Many physicians believe there is a need to identify additional markers to increase the sensitivity and specificity of tests used to identify individuals that are at risk for fatal arrhythmias.

### BRIEF SUMMARY OF THE INVENTION

The present invention is a method and kits for classifying individuals based on susceptibility to sudden cardiac death. One or more single nucleotide polymorphisms are identified in one or more genes whose products are involved in the cardiac action potential.

### BRIEF DESCRIPTION OF THE DRAWINGS

FIG. 1 is a graph illustrating the probability of experiencing a fatal arrhythmia as a function of allele specific inheritance of SNP rs1009531.

FIG. 2 is a graph illustrating the probability of experiencing a fatal arrhythmia as a function of allele specific inheritance of SNP rs1428568.

FIG. 3 is a graph illustrating the probability of experiencing a fatal arrhythmia as a function of allele specific inheritance of SNP rs918050.

FIG. 4 is a graph illustrating the probability of experiencing a fatal arrhythmia as a function of allele specific inheritance of SNP rs198544.

FIGS. 5a, 5b, and 5c are graphs illustrating the probability of experiencing a fatal arrhythmia as a function of allele specific inheritance of SNPs rs1009531 and rs1428568.

FIG. 6 is a tree analysis using genetic and demographic class identifiers for classification.

FIG. 7 is a graph illustrating the probability of experiencing a fatal arrhythmia as a function of allele specific inheritance of SNP rs1008832.

FIG. 8 is a graph illustrating the probability of experiencing a fatal arrhythmia as a function of allele specific inheritance of SNP rs2238043.

FIG. 9 is a graph illustrating the probability of experiencing a fatal arrhythmia as a function of allele specific inheritance of SNP rs198544.

FIG. 10 is a graph illustrating the probability of experiencing a fatal arrhythmia as a function of allele specific inheritance of SNP rs1009531.

FIG. 11 is a graph illustrating the probability of experiencing a fatal arrhythmia as a function of allele specific inheritance of SNP rs2121081.

FIG. 12 is a graph illustrating the probability of experiencing a fatal arrhythmia as a function of allele specific inheritance of SNP rs1428568.

FIG. 13 is a graph illustrating the probability of experiencing a fatal arrhythmia as a function of allele specific inheritance of SNP rs918050.

FIG. 14 is a graph illustrating the probability of experiencing a fatal arrhythmia as a function of allele specific inheritance of SNP rs1483312.

FIG. 15 is a graph illustrating the probability of experiencing a fatal arrhythmia as a function of allele specific inheritance of SNP rs1859037.

FIG. 16 is a graph illustrating the probability of experiencing a fatal arrhythmia as a function of allele specific inheritance of SNP rs6964587.

FIG. 17 is a graph illustrating the probability of experiencing a fatal arrhythmia as a function of allele specific inheritance of SNPs rs2238043 and rs1483312.

### DESCRIPTION

To identify candidate SNPs for use in classifying individuals based on susceptibility to SCD, a list of genes was generated whose products are known to be involved in the formation of the cardiac action potential. The genes included expressed products that are major factors involved in Long QT Syndrome or other cardiac disorders such as Andersen syndrome, and other functional products involved in the cardiac action potential.

Next, a list of candidate SNPs was generated for further evaluation. Candidate SNPs were identified within the above genes from public databases. The list of candidate SNPs was further narrowed. Sequencing errors in the human genome have resulted in a tremendous amount of non-polymorphic SNPs in the public databases. To avoid these, SNPs were selected that have been experimentally validated, that are listed in two or more public/private databases and, most importantly, that contain allele frequency information. Whenever possible, minor allele frequency data from Caucasian populations was recovered as will become evident below. The resulting SNP list was prioritized, and 200 SNPs were chosen for evaluation from patient samples. 186 of these 200 SNPs were read from DNA samples obtained from 81 patients, while the remaining 14 SNPs could not be read due to process failures with primers and instrumentation. However, SNPs that were not chosen may also be useful in classifying individuals. The decision not to evaluate all SNPs is not an indication that the SNPs which were not chosen are not SCD-associated polymorphisms.

To evaluate the chosen 186 SNPs, a traditional case-control trial was designed to gather genetic samples from patients with myocardial infarction. Table 2 summarizes the study design.

TABLE 2

| | | | |
|---|---|---|---|
| Test | CAD(+) | ICD(+) | One sustained VT/VF episode with |
| | | ATP(+) | cycle length ≦ 400 ms |
| | | Recent Shock(−) | |
| Control | CAD(+) | ICD(−) | No History of VT/VF |

CAD: Coronary Artery Disease
ICD: Implantable Defibrillator
ATP: Anti-Tachy Pacing
VT/VF: Ventricular Tachycardia/Ventricular Fibrillation

DNA was extracted from tissue samples of 90 patients to obtain genetic information. However, nine patients were excluded, because their recorded arrhythmic events disqualified them from this case arm. The patient population was chosen to be as uniform as possible (e.g. Caucasian, male, etc.). Tissue samples can be of any type such as blood, skin, etc. Genetic information may also be obtained from sequence data in the form of electronic, print, or any other recorded media. DNA extraction may be performed using any of a number of techniques including phenol-chloroform extraction, phenol-chloroform extraction followed by ethanol precipitation or isopropanol precipitation of DNA, glass bead purification, or salt precipitation (See Current Protocols in Molecular Biology, Published by John Willey & Sons, updated annually and Miller, S. A., Dykes, D. D. and Polesky, H. F. (1988), Nucleic Acids Res 16(3):1215). DNA extraction kits from commercial vendors such as Qiagen and Stratagene may also be used.

The genetic sequence for each patient was discerned. Sequence information may be obtained by any of a number of ways such as the Sanger sequencing technique following amplification by PCR, DNA microarray chips containing 25-mer oligos, SNP stream sequencing, bead arrays (e.g. AmpaSand SIFT™), mass spectrometry (sequenome), fragment analysis using capillary electrophoresis, and Taqman Allelic Discrimination Assay.

In the present study, SNP stream sequencing with primer extension was utilized. Three primers were used for each SNP assay. Primers upstream and downstream from each polymorphic site are used to amplify the SNPs along with approximately 100-150 base-pair flanking regions. A SNP primer is designed to anneal adjacent to the polymorphic site and is extended by a single terminating base to query the SNP site. Each SNP primer includes a 20 nucleotide capture tag followed by a 25 nucleotide SNP specific region. By using 12 different capture tags, the system is multiplexed with up to 12 SNPs per well.

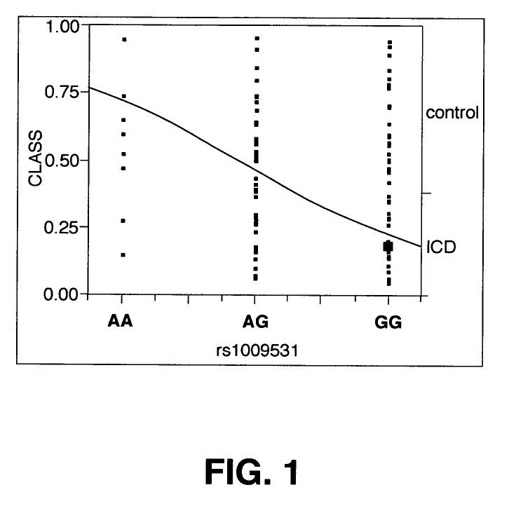

186 pairs of SNP alleles were read for each sample, and a statistical analysis was carried out using JMP software (SAS Institute Inc., Cary, N.C., USA) to determine the relationship between the inheritance patterns and the penetration of the ventricular tachycardia phenotype. Four of the 186 SNPs showed significant ability to classify the patients into high and low risk for sudden cardiac death. FIGS. 1-4 illustrate the results of the statistical analysis. As used throughout, a represents adenine, t represents thymine, c represents cytosine, and g represents guanine.

FIG. 1 is a graph showing the probability of experiencing a fatal arrhythmia as a function of the allele specific inheritance pattern of SNP rs1009531. SNP rs1009531 and its flanking regions were amplified using the following primers: 5′-tag acg gaa gta aag gtt aga tcc-3′ (SEQ ID NO. 1) and 5′-tgt gtt tgg tgt ggg cag-3′ (SEQ ID NO. 2). The SNP site was sequenced using the following oligomer: 5′-gtg att ctg tac gtg tcg cct ttt gcc ttt cct cac aga gct tgg-3′ (SEQ ID NO. 3).

SNP rs1009531 is located at position chr1:112082919, Build 123, within the KCND3 gene (Accession No. NT_{—}019273). The position information provides the chromosome and nucleotide position, and the Build references the sequence update information from which the SNP position was obtained. The most updated information is given, however, as genome information is generated, the position may change slightly. KCND3 codes for the potassium voltage-gated channel, Shal-related subfamily, member 3 protein. The product is a member of the potassium channel, voltage-gated, Shal-related subfamily, which form voltage-activated A-type potassium ion channels and are important in the repolarization phase of the action potential. This member, member 3, includes two isoforms with different sizes that are encoded by alternatively spliced transcript variants of the gene.

The horizontal axis of the graph lists the possible genotypes at this particular SNP. The vertical axis is the probability of experiencing SCD. The sequencing results of each patient were plotted, and the trace indicates the results. Data points below the trace represent test patients having ICDs, and points above the trace represent control patients.

As shown by the graph in FIG. 1, the presence of t at the SNP position indicates increased susceptibility to SCD as compared to the presence of c at the SNP position. Conversely, the presence of c at the SNP position indicates decreased susceptibility as compared to the presence of t. Specifically, individuals having the t/t genotype have about a 75% probability of experiencing SCD, while the t/c genotype indicates about a 50% probability, and the c/c genotype indicates about a 30% probability of experiencing SCD.

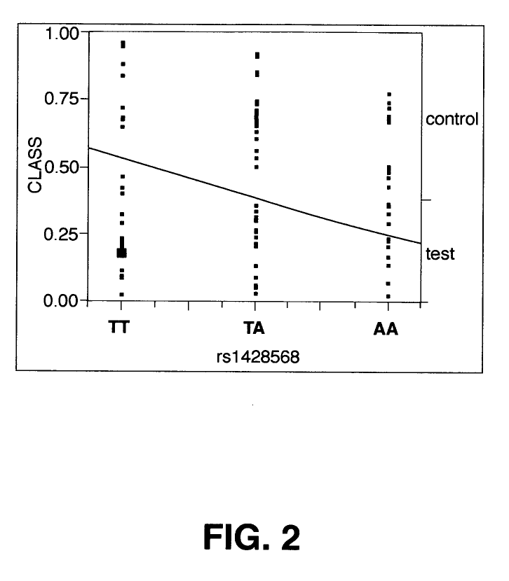

FIG. 2 is a graph showing the probability of experiencing a fatal arrhythmia as a function of the allele specific inheritance pattern of SNP rs1428568. SNP rs1428568 and its flanking regions were amplified using the following primers: 5′-act cta aaa aat cat gtg cca gc-3′ (SEQ ID NO. 4) and 5′-ttg tgg aac tgg cac tgg-3′ (SEQ ID NO. 5). The SNP site was sequenced using the following oligo: 5′-cga ctg tag gtg cgt aac tct cca gct aat gtt tgc cct ctt ctc-3′ (SEQ ID NO. 6).

SNP rs1428568 is located at position chr2:40296347, Build 123, within the SLC8A1 gene (Accession No. NT_{—}022184), which codes for the solute carrier family 8 (sodium/calcium exchanger), member 1 protein. The protein is a sodium/calcium exchanger that is the primary mechanism by which Ca²⁺ is released from cardiac myocytes during relaxation. In the heart, this protein may play a key role in the action of digitalis and is the dominant mechanism in returning cardiac myocytes to their resting state following excitation.

Here again, the horizontal axis lists the possible genotypes for this particular SNP. The vertical axis is the probability of experiencing SCD. The data points and resulting trace are as described for FIG. 1.

The presence of t at the SNP position classifies individuals as having increased susceptibility to SCD as compared to the presence of a at the SNP position. Conversely, the presence of a at the SNP position classifies individuals as having decreased susceptibility to sudden cardiac death as compared to the presence of t. Specifically, individuals having the t/t genotype have about a 60% probability of experiencing SCD, while the t/a genotype indicates about a 45% probability, and the a/a genotype indicates about a 30% probability of experiencing SCD.

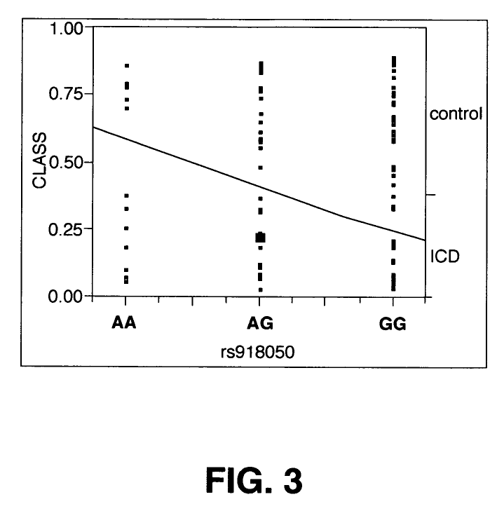

FIG. 3 is a graph showing the probability of experiencing a fatal arrhythmia as a function of the allele specific inheritance pattern of SNP rs918050. SNP rs918050 and its flanking regions were amplified using the following primers: 5′-cct gca aag ctt tcc cgta-3′ (SEQ ID NO. 7) and 5′-cta gaa cat gag caa ata ctt aat taa-3′ (SEQ ID NO. 8). The SNP site was sequenced using the following oligo: 5′-aga tag agt cga tgc cag cta tgg tac aat taa gtt taa ctt aca-3′ (SEQ ID NO. 9).

SNP rs918050 is located at position chr2:40331321, Build 116, within the SLC8A1 gene, which codes for the solute carrier family 8 (sodium/calcium exchanger), member 1 protein. This protein was summarized above. The graph is as described above except that the specific alleles at this SNP are t and c.

The presence of t at position the SNP position classifies individuals as having increased susceptibility to SCD as compared to the presence of c at the SNP position. Conversely, the presence of c at the SNP position classifies individuals as having decreased susceptibility to SCD as compared to the presence of t. Specifically, individuals having the t/t genotype have about a 60% probability of experiencing SCD, while the t/c genotype indicates about a 45% probability, and the c/c genotype indicates about a 25% probability of experiencing SCD.

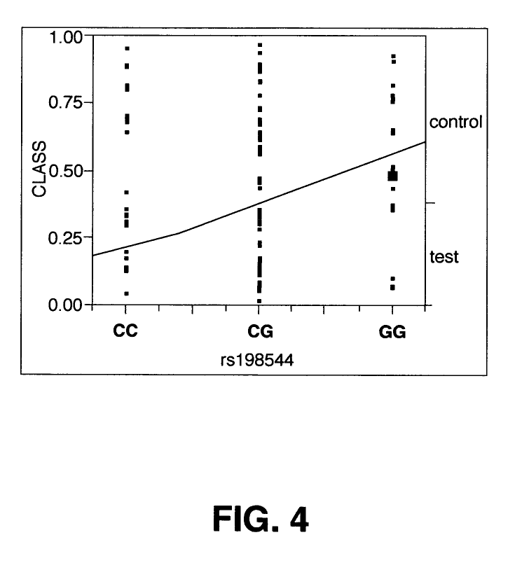

FIG. 4 is a graph showing the probability of experiencing a fatal arrhythmia as a function of the allele specific inheritance pattern of SNP rs198544. SNP rs198544 and its flanking regions were amplified using the following primers: 5′-cct ggc act agg tgt aag gc-3′ (SEQ ID NO. 10) and 5′-gag gct ggt ggt gga aga-3′ (SEQ ID NO. 11). The SNP site was sequenced using the following oligo: 5′-cgt gcc gct cgt gat aga atg cag acg tcc aca gct gca gtc ccc-3′ (SEQ ID NO. 12).

SNP rs198544 is located at position chr17:46000504, Build 120, within the CACNA1G gene (Accession No. NT_{—}010783), which codes for the calcium channel, voltage-dependent, alpha 1G subunit. This is a low-voltage-activated calcium channel referred to as a “T-type” channel, because its currents are transient and tiny. T-type channels are thought to be involved in pacemaker activity, low-threshold calcium spikes, neuronal oscillations and resonance, and rebound burst firing. The graph is as described above except that the specific alleles at this SNP are g and c.

The presence of g at the SNP position classifies individuals as having increased susceptibility to SCD as compared to the presence of c at the SNP position. Conversely, the presence of c at the SNP position classifies individuals as having decreased susceptibility to SCD as compared to the presence of g. Specifically, individuals having the g/g genotype have about a 60% probability of experiencing SCD, while the g/c genotype indicates about a 45% probability, and the c/c genotype indicates about a 25% probability of experiencing SCD.

Each SNP described above can be used individually to predict an individual's probability of experiencing SCD. In turn, this will also provide a better indication as to whether an individual will benefit from an IMD or to identify the best drug regimen for the individual. The small “p-values” shown with each graph indicate the certainty of each SNP's predictive power. These tests may be used alone or in combination with other physiological, demographical, proteomic, and/or lipidomic identifiers, collectively referred to as class identifiers, for classification. For example, the test for SNP rs1009531 may be combined with an ejection fraction test to predict whether an individual will benefit from implantation of an IMD. Sensitivity and specificity of the test can be further increased by additional tests, such as T-wave alternans. Alternatively, as shown below, one or more SNP tests may be combined to improve the predictive power.

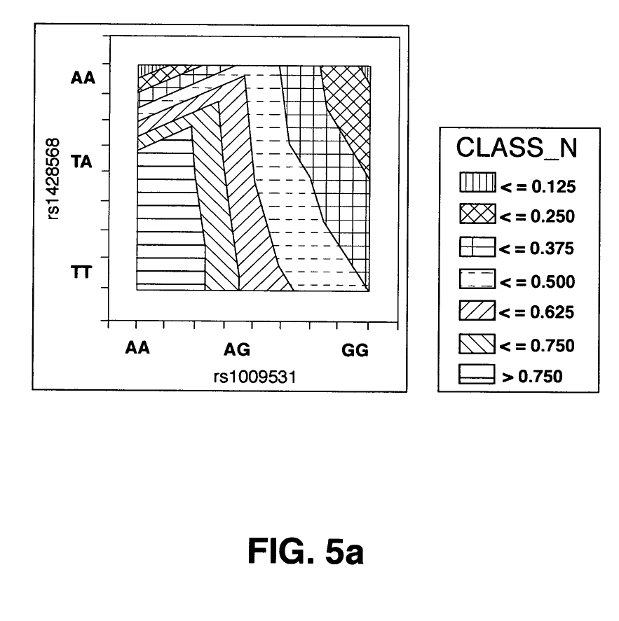

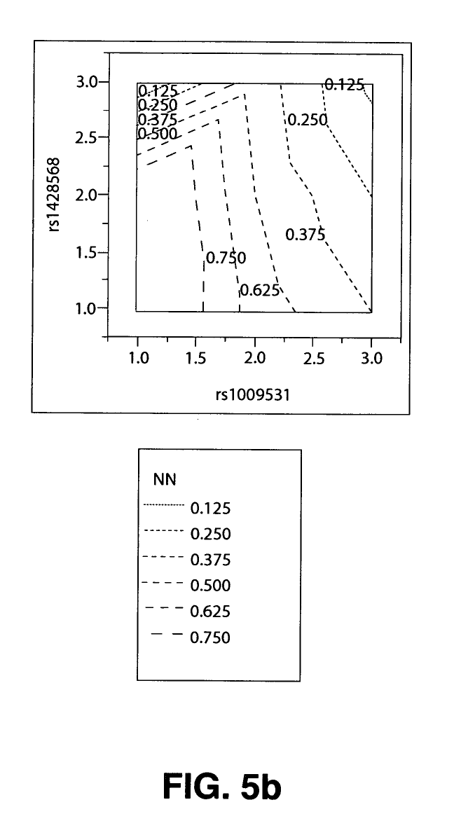

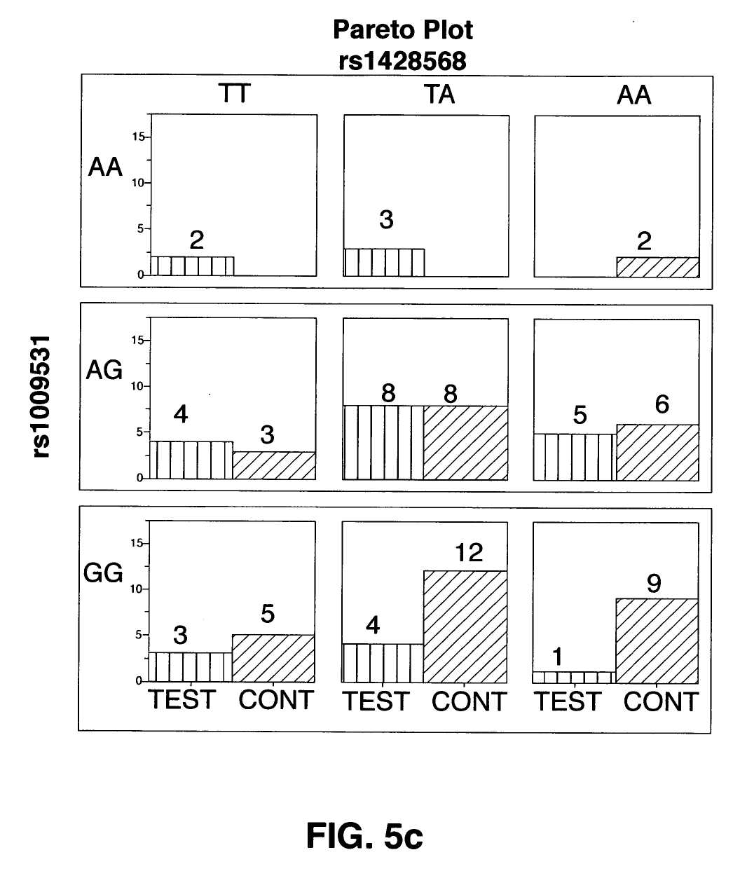

FIGS. 5a, 5b, and 5c are graphs showing the probability of experiencing SCD as a function of the allele specific inheritance pattern of SNPs rs1009531 and rs1428568. Data from 75 of the same 90 patients was used.

FIGS. 5a and 5b are contour plots. The horizontal axes are the possible genotypes of SNP rs1009531, and the vertical axes are the possible genotypes of SNP rs1428568. Matrixes were formed where the points of intersection are the points of interest. The boxes next to the graphs in FIG. 5a identify the probabilities that correspond to the intersection points.

FIG. 5c is a Pareto Plot. The solid bars represent the test patients, and the grid-patterned bars represent control patients. Here, the horizontal axes are SNP rs1428568 genotypes, and the vertical axes are SNP rs1009531 genotypes. FIGS. 5a, 5b, and 5c illustrate different ways of presenting the same information.

Most genotype combinations provide probabilities that further stratify the classification method. For example, genotype profiles of t/t-t/t and t/t-t/a have a greater than 75% probability of experiencing a fatal arrhythmia. t/t-a/a and c/c-a/a have a probability less than or equal to 25%. t/c-t/t, t/c-t/a, and t/c-a/a all have a probability less than or equal to 62.5%, and therefore, the specificity is not improved for these patients. A c/c-t/t genotype profile has a probability less than or equal to 50%, while a c/c-t/a genotype profile has a probability less than or equal to 37.5%. Combining these SNP tests particularly improves the predictive power for individuals heterozygous at SNP rs1428568.

A second representative clinical study was carried out to discover, among other things, genetic class identifiers for classifying patients as to risk of ventricular tachycardia/ventricular fibrillation (VT/VF) or SCD. These patients also had coronary artery disease and met specific inclusion criteria. They were divided into test and control groups based on whether or not patients had an IMD. The patients having an IMD also had at least one true VT/VF episode with a cycle length less than or equal to 400 ms terminated in the last 90 days. A total of 29 patients were in the test group, which consists of patients with an IMD, and 49 patients were in the control group. Patients from the first study described above were also part of this second study.

Patients filled out an extensive questionnaire that included medical information, and genetic information was collected as described above. DNA from each patient was extracted from cells in the blood samples. There were 162 gene SNPs from which genotypes were obtained for the patients.

A chi-square test was performed using the response variable (test, control) and each of the 162 SNP variables. Table 3 lists SNPs and resulting p-values identified as correlating genotypes to patient groups that were not independent.

TABLE 3

| Gene SNP | # of Genotypes | P-value |
|---|---|---|
| rs2239507 | 3 | 0.0051 |
| rs7626962 | 2 | 0.0094* |
| rs3743496 | 3 | 0.012* |
| rs723672 | 6 | 0.0060* |
| rs2072715 | 2 | 0.0473* |
| rs12276475 | 3 | 0.0092* |
| rs1544503 | 3 | 0.0349* |
| rs3752158 | 3 | 0.0465* |
| rs1023214 | 3 | 0.0322* |
| rs730818 | 3 | 0.0372 |
| rs1842082 | 3 | 0.0434 |
| rs7578438 | 5 | 0.0443* |
| rs545118 | 3 | 0.0415* |
| rs802351 | 5 | 0.0314* |

*Fisher's exact test used, as criteria for chi-square test not met

In some cases the expected cell counts were too small, and it was determined that the chi-square test was not reliable. Instead, Fisher's exact test was used. Fourteen SNPs produced p-values less than 0.05.

More detail regarding each SNP is provided below. In some cases, SNPs could not be determined from some samples, and in these cases, the number of missing samples is indicated as “Frequency Missing”.

SNP rs2239507 is located at position chr12:5021395, Build 123, within the KCNA5 gene (Accession No. NT_{—}009759), which codes for the potassium voltage-gated channel, subfamily A, member 5 protein. This protein mediates the voltage-dependent potassium ion permeability of excitable membranes. rs2239507 and its flanking regions were amplified using the following primers: 5′-agt cag gat cag gta ttt ttc ct-3′ (SEQ ID NO. 13) and 5′-aga acc cag gtg aac caa t-3′ (SEQ ID NO. 14). The SNP site was sequenced using the following oligo: 5′-aga tag agt cga tgc cag ctt cat ggg tct ctg acc tca ctg tct-3′ (SEQ ID NO. 15). Table 4 includes the statistical breakdown of allele distribution within the patient groups.

TABLE 4

| Patient | SNP rs2239507 | | | |
|---|---|---|---|---|
| Group | g/g | g/t | t/t | Total |
| Test (N, %) | 12 | 7 | 10 | 29 |
| | 41.4% | 24.1% | 34.5% | 100% |
| Control | 5 | 21 | 23 | 49 |
| (N, %) | 10.2% | 42.9% | 46.9% | 100% |
| Total (N, %) | 17 | 28 | 33 | 78 |
| | 21.8% | 35.9% | 42.3% | 100% |

As shown in Table 4, twelve, or 41.4%, of test patients had the genotype g/g, whereas only five, or 10.2%, of control patients had the genotype g/g.

rs7626962 is located at position chr3:38595911, Build 116, within the SCN5A gene (Accession No. NT_{—}022517). The SCN5A gene codes for the sodium channel, voltage-gated, type V, alpha (long QT syndrome 3) protein. This protein produces sodium channels that transport sodium ions across cell membranes and plays a key role in generating and transmitting electrical signals. rs7626962 and its flanking regions were amplified using the following primers: 5′-cct ccg gat tcc agg acc-3′ (SEQ ID NO. 16) and 5′-ttc cgc ttt cca ctg ctg-3′ (SEQ ID NO. 17). The SNP site was sequenced using the following oligo: 5′-gga tgg cgt tcc gtc cta tta gat gca ctg gcc tcg gcc tca gag-3′ (SEQ ID NO. 18). Table 5 shows the statistical breakdown of the patient groups.

TABLE 5

| | |
|---|---|
| Patient | SNP rs7626962 |

| Group | g/g | t/t | Total |
|---|---|---|---|
| Test (N, %) | 23 | 6 | 29 |
| | 79.3% | 20.7% | 100% |
| Control | 48 | 1 | 49 |
| (N, %) | 98.0% | 2.0% | 100% |
| Total (N, %) | 71 | 7 | 78 |
| | 91.0% | 9.0% | 100% |

rs3743496 is located at position chr15:71401461, Build 121, within the gene HCN4 (Accession No. NT_{—}010194). The gene codes for the potassium/sodium hyperpolarization-activated cyclic nucleotide-gated channel 4 protein. The protein is a hyperpolarization-activated ion channel that may contribute to native pacemaker currents in the heart. rs3743496 and its flanking regions were amplified using the following primers: 5′-att gtg ttc att tag aga aac agc t-3′ (SEQ ID NO. 19) and 5′-ctt ctg agg ctc ccc agg-3′ (SEQ ID NO. 20). The SNP site was sequenced using the following oligo: 5′-agg gtc tct acg ctg acg atc tgt aac ttg gag ctc cac tct gcc-3′ (SEQ ID NO. 21). Table 6 shows the statistical breakdown of the patient groups.

TABLE 6

| Patient | SNP rs3743496 | | | |
|---|---|---|---|---|
| Group | a/a | c/a | c/c | Total |
| Test (N, %) | 2 | 3 | 24 | 29 |
| | 6.9% | 10.3% | 82.8% | 100% |
| Control | 0 | 0 | 48 | 48 |
| (N, %) | 0.0% | 0.0% | 100% | 100% |
| Total (N, %) | 2 | 3 | 72 | 77 |
| | 2.6% | 3.9% | 93.5% | 100% |

Frequency Missing = 1

rs2072715 is located at position chr22:38339084, Build 121, within the gene CACNA1I (Accession No. NT_{—}011520). The gene codes for the calcium channel, voltage-dependent, alpha 11 subunit protein. The protein is a voltage-sensitive calcium channel that serves pacemaking functions in central neurons and cardiac nodal cells. rs2072715 and its flanking regions were amplified using the following primers: 5′-tca gta gga aat gaa ggc ttt t-3′ (SEQ ID NO. 22) and 5′-att tca ggg aac gaa tgg a-3′ (SEQ ID NO. 23). The SNP site was sequenced using the following oligo: 5′-gcg gta ggt tcc cga cat att ctt caa gca gcg gga ggg ggt ggc-3′ (SEQ ID NO. 24). Table 7 includes the statistical breakdown of the allelic distribution within the patient groups.

TABLE 7

| | |
|---|---|
| Patient | SNP rs2072715 |

| Group | g/a | g/g | Total |
|---|---|---|---|
| Test (N, %) | 8 | 21 | 29 |
| | 27.6% | 72.4% | 100% |
| Control | 4 | 45 | 49 |
| (N, %) | 8.2% | 91.8% | 100% |
| Total (N, %) | 12 | 66 | 78 |
| | 15.4% | 84.6% | 100% |

rs12276475 is located at position chr11:29989733, Build 120, within the KCNA4 gene (Accession No. NT_{—}009237). This gene codes for the potassium voltage-gated channel, shaker-related subfamily, member 4 protein. This protein mediates the voltage-dependent potassium ion permeability of excitable membranes. rs12276475 and its flanking regions were amplified using the following primers: 5′-aca aac tca aag gaa aac cat aca-3′ (SEQ ID NO. 25) and 5′-atc tcg tca tgg cac tga gt-3′ (SEQ ID NO. 26). The SNP site was sequenced using the following oligo: 5′-gtg att ctg tac gtg tcg cct ggg ttg ttg aat gat act tca gca-3′ (SEQ ID NO. 27). Table 8 includes the statistical breakdown for the patient groups.

TABLE 8

| Patient | SNP rs12276475 | | | |
|---|---|---|---|---|
| Group | a/a | c/a | c/c | Total |
| Test (N, %) | 11 | 18 | 0 | 29 |
| | 37.9% | 62.1% | 0.0% | 100% |
| Control | 33 | 15 | 1 | 49 |
| (N, %) | 67.3% | 30.6% | 2.1% | 100% |
| Total (N, %) | 44 | 33 | 1 | 78 |
| | 56.4% | 42.3% | 1.3% | 100% |

rs1544503 is located at position chr12:2308902, Build 123, within the CACNA1C gene (Accession No. NT_{—}009759). The function of the gene product was discussed above. rs1544503 and its flanking regions were amplified using the following primers: 5′-aat agg atg cac ttg ctt gac-3′ (SEQ ID NO. 28) and 5′-atg agg aag agt ccc ttc acc-3′ (SEQ ID NO. 29). The SNP site was sequenced using the following oligo: 5′-agg gtc tct acg ctg acg att gat gtt cat tga tgg gga cag gca-3′ (SEQ ID NO. 30). Table 9 shows the statistical breakdown.

TABLE 9

| Patient | SNP rs1544503 | | | |
|---|---|---|---|---|
| Group | c/c | t/c | t/t | Total |
| Test (N, %) | 16 | 7 | 5 | 28 |
| | 57.1% | 25.0% | 17.9% | 100% |
| Control | 21 | 24 | 2 | 47 |
| (N, %) | 44.7% | 51.1% | 4.3% | 100% |
| Total (N, %) | 37 | 31 | 7 | 75 |
| | 49.3% | 41.3% | 9.3% | 100% |

Frequency Missing = 3

rs723672 is located at position chr12:2031822, Build 120, within of the CACNA1C gene (Accession No. NT_{—}009759). The function of the gene product was discussed previously. rs723672 and its flanking regions were amplified using the following primers: 5′-tat ctg tca ctt cta caa ccg ct-3′ (SEQ ID NO. 31) and 5′-aat tcc aag gag gag gaa tac a-3′ (SEQ ID NO. 32). The SNP site was sequenced using the following oligo: 5′-gcg gta ggt tcc cga cat ata tcg ggc cac tga aca aaa cgg caa-3′ (SEQ ID NO. 33). Table 10 shows the statistical breakdown.

TABLE 10

| | |
|---|---|
| Patient | SNP rs723672 |

| Group | a/a | g/a | g/g | Total |
|---|---|---|---|---|
| Test | 2 | 15 | 11 | 28 |
| (N, %) | 7.1% | 53.6% | 39.3% | 100% |
| Control | 13 | 15 | 19 | 47 |
| (N, %) | 27.7% | 31.9% | 40.4% | 100% |
| Total | 15 | 30 | 30 | 75 |
| (N, %) | 20.0% | 40.0% | 40.0% | 100% |

Frequency Missing = 3

rs3752158 is located at position chr19:558984, Build 120, within the HCN2 gene (Accession No. NT_{—}011255). This gene codes for the hyperpolarization activated cyclic nucleotide-gated potassium channel 2 protein. This protein may play a role in generation of neuronal pacemaker activity. rs3752158 and its flanking regions were amplified using the following primers: 5′-atg cac agc atg tgg ctc-3′ (SEQ ID NO. 34) and 5′-tga gca cct gcc cac cac-3′ (SEQ ID NO. 35). The SNP site was sequenced using the following oligo: 5′-agg gtc tct acg ctg acg att gca gaa cca ctc gtg gag tga act-3′ (SEQ ID NO. 36). Table 11 shows a statistical breakdown of the allelic distribution.

TABLE 11

| Patient | SNP rs3752158 | | | |
|---|---|---|---|---|
| Group | c/c | c/g | g/g | Total |
| Test (N, %) | 5 | 2 | 20 | 27 |
| | 18.5% | 7.4% | 74.1% | 100% |
| Control | 1 | 7 | 40 | 48 |
| (N, %) | 2.1% | 14.6% | 83.3% | 100% |
| Total (N, %) | 6 | 9 | 60 | 75 |
| | 8.0% | 12.0% | 80.0% | 100% |

Frequency Missing = 3

rs1023214 is located at position chr1:234128106, Build 123, within the RYR2 gene (Accession No. NT_{—}004836). This gene codes for the ryanodine receptor 2 protein. The protein is involved in providing calcium required for cardiac muscle excitation-contraction coupling. rs1023214 and its flanking regions were amplified using the following primers: 5′-agt aaa gca tta tgg agg cat aaa-3′ (SEQ ID NO. 37) and 5′-gca tct aag ttc tcc taa att ttt tat t-3′ (SEQ ID NO. 38). The SNP site was sequenced using the following oligo: 5′-ggc tat gat tcg caa tgc ttg ttt gga tta tga cat cat tct ata-3′ (SEQ ID NO. 39). Table 12 shows a statistical breakdown of the allelic distribution.

TABLE 12

| Patient | SNP rs1023214 | | | |
|---|---|---|---|---|
| Group | a/a | g/a | g/g | Total |
| Test (N, %) | 16 | 3 | 3 | 22 |
| | 72.7% | 13.6% | 13.6% | 100% |
| Control | 15 | 15 | 2 | 32 |
| (N, %) | 46.9% | 46.9% | 6.3% | 100% |
| Total (N, %) | 31 | 18 | 5 | 54 |
| | 57.4% | 33.3% | 9.3% | 100% |

Frequency Missing = 24

rs730818 is located at position chr17:40227887, Build 86, within the GJA7 gene (Accession No. NT_{—}010783). The gene codes for the gap junction alpha-7 (Connexin 45) protein. This protein is involved in cell-to-cell communication. rs730818 and its flanking regions were amplified using the following primers: 5′-ttt ttc ttt cag aag ccc ct-3′ (SEQ ID NO. 40) and 5′-aca tga cat ggt gac aag ca-3′ (SEQ ID NO. 41). The SNP site was sequenced using the following oligo: 5′-cgt gcc gct cgt gat aga att ctt tgt caa ttg act ttt tct ccc-3′ (SEQ ID NO. 42). Table 13 includes a statistical breakdown of the allelic distribution.

TABLE 13

| Patient | SNP rs730818 | | | |
|---|---|---|---|---|
| Group | a/a | g/a | g/g | Total |
| Test (N, %) | 0 | 16 | 9 | 25 |
| | 0.0% | 64.0% | 36.0% | 100% |
| Control | 10 | 21 | 18 | 49 |
| (N, %) | 20.4% | 42.9% | 36.7% | 100% |
| Total (N, %) | 10 | 37 | 27 | 74 |
| | 13.5% | 50.0% | 36.5% | 100% |

Frequency Missing = 4

rs1842082 is located at position chr1:233951526, Build 123, within the RYR2 gene (Accession No. NT_{—}004836). This gene codes for the gap junction alpha-7 protein as described above. rs1842082 and its flanking regions were amplified using the following primers: 5′-aag aaa aaa tac aaa gac agt ggc-3′ (SEQ ID NO. 43) and 5′-tgt caa aac ctt tgg ttc aaa-3′ (SEQ ID NO. 44). The SNP site was sequenced using the following oligo: 5′-agg gtc tct acg ctg acg att tgt taa gcc tcc ttc ccg tta ttc-3′ (SEQ ID NO. 45). Table 14 is a statistical breakdown of the patient groups.

TABLE 14

| Patient | SNP rs1842082 | | | |
|---|---|---|---|---|
| Group | c/c | c/g | g/g | Total |
| Test (N, %) | 5 | 19 | 5 | 29 |
| | 17.2% | 65.5% | 17.2% | 100% |
| Control | 21 | 19 | 9 | 49 |
| (N, %) | 42.9% | 38.8% | 18.4% | 100% |
| Total (N, %) | 26 | 38 | 14 | 78 |
| | 33.3% | 48.7% | 18.0% | 100% |

rs545118 is located at position chr11:30002105, Build 123, within the KCNA4 gene (Accession No. NT_{—}009237). The gene product was described previously. rs545118 and its flanking regions were amplified using the following primers: 5′-cat ttt tac aga caa gaa aat tta gg-3′ (SEQ ID NO. 46) and 5′-ata ggt ttt tct ctc cag cc-3′ (SEQ ID NO. 47). The SNP site was sequenced using the following oligo: 5′-acg cac gtc cac ggt gat ttc ttg gct gca gaa cct ctg gct aag-3′ (SEQ ID NO. 48). Table 15 shows the statistical breakdown of the patient groups.

TABLE 15

| Patient | SNP rs545118 | | | |
|---|---|---|---|---|
| Group | a/a | t/a | t/t | Total |
| Test (N, %) | 19 | 10 | 0 | 29 |
| | 65.5% | 34.5% | 0.0% | 100% |
| Control | 20 | 24 | 5 | 49 |
| (N, %) | 40.8% | 49.0% | 10.2% | 100% |
| Total (N, %) | 39 | 34 | 5 | 78 |
| | 50.0% | 43.6% | 6.4% | 100% |

rs7578438 is located at position chr2:155388192, Build 121, within the KCNJ3 gene (Accession No. NT_{—}005403). This gene codes for the potassium inwardly-rectifying channel, subfamily J, member 3 protein. This protein forms potassium channels that are characterized by a greater tendency to allow potassium to flow into the cell rather than out of it. rs7578438 and its flanking regions were amplified using the following primers: 5′-cat aaa tct taa ctt tta gcg atc g-3′ (SEQ ID NO. 49) and 5′-agg atc gtt ttc tag ata caa atg tat aa-3′ (SEQ ID NO. 50). The SNP site was sequenced using the following oligo: 5′-agc gat ctg cga gac cgt atg ata tgg tct aga tca aca ata att-3′ (SEQ ID NO. 51). Table 14 includes a statistical breakdown of the patient groups.

TABLE 16

| | |
|---|---|
| Patient | SNP rs7578438 |

| Group | g/g | g/t | t/t | Total |
|---|---|---|---|---|
| Test | 9 | 16 | 2 | 27 |
| (N, %) | 33.3% | 59.3% | 7.4% | 100% |
| Control | 14 | 29 | 6 | 49 |
| (N, %) | 28.6% | 59.2% | 12.2% | 100% |
| Total | 23 | 45 | 8 | 76 |
| (N, %) | 30.3% | 59.2% | 10.5% | 100% |

Frequency Missing = 2

rs802351 is located at position chr7:119166671, Build 123, within the KCND2 gene (Accession No. NT_{—}007933). This gene codes for the potassium voltage-gated channel, subfamily D, member 2 protein. This protein is a pore-forming subunit of voltage-gated, rapidly inactivating A-type potassium channels. rs802351 and its flanking regions were amplified using the following primers: 5′-aaa act ata agt att ttc ttg tga agg tg-3′ (SEQ ID NO. 52) and 5′-aac att tgc caa tgc aat g-3′ (SEQ ID NO. 53). The SNP site was sequenced using the following oligo: 5′-gga tgg cgt tcc gtc cta tta gca gca ttt aaa taa atg ccc tct-3′ (SEQ ID NO. 54). Table 17 includes a statistical breakdown of the allelic distribution.

TABLE 17

| | |
|---|---|
| Patient | SNP rs802351 |

| Group | g/g | g/t | t/t | Total |
|---|---|---|---|---|
| Test | 1 | 10 | 17 | 28 |
| (N, %) | 3.6% | 35.7% | 60.7% | 100% |
| Control | 2 | 9 | 38 | 49 |
| (N, %) | 4.1% | 18.4% | 77.6% | 100% |
| Total | 3 | 19 | 55 | 77 |
| (N, %) | 3.9% | 24.7% | 71.4% | 100% |

Frequency Missing = 1

Using the same data, a second analysis was performed to compare major and minor allele frequencies among the patient groups. The major allele is that which occurs most frequently, while the minor allele is that which occurs least frequently. Each patient has two alleles, therefore, 156 alleles were assessed for each SNP. It was subsequently determined whether patients with particular alleles in their genotype were more likely to belong to one group over the other.

Table 18 shows the results of either a chi-square or Fisher's exact test analysis where at least one of the alleles resulted in a p-value less than 0.05.

TABLE 18

| Gene/Accession No. | SNP | Major Allele | P-value | Minor Allele | P-value |
|---|---|---|---|---|---|
| Refer to discussion | rs12276475 | a | 0.4388 | C (test) | 0.0113 |
| KCNK4 | rs1320840 | g | 0.6372 | a (control) | 0.0293 |
| NT_033903 | | | | | |
| KCNA4 | rs1323860 | a (test) | 0.022 | g | 0.4671 |
| NT_009237 | | | | | |
| KCND3 | rs1538389 | c (control) | 0.0448* | t | 0.3341 |
| NT_019273 | | | | | |
| KCND3 | rs1808973 | t | 0.6998 | c (control) | 0.0443 |
| NT_019273 | | | | | |
| Refer to discussion | rs1842082 | c | 0.9003 | g (test) | 0.0204 |
| KCND2 | rs1859534 | t (test) | 0.7039 | a | 0.0317 |
| NT_007933 | | | | | |
| Refer to discussion | rs2072715 | g | N/A** | a (test) | 0.0473* |
| Refer to discussion | rs2238043 | g (test) | 0.0462* | a | 0.142 |
| Refer to discussion | rs2239507 | t (control) | 0.0013 | g | 0.2819 |
| SLC8A1 | rs2373860 | t (control) | 0.0419 | a | 0.7046 |
| NT_022184 | | | | | |
| KCNK4 | rs3739081 | a (control) | 0.0368 | g | 0.7621 |
| NT_033903 | | | | | |
| Refer to discussion | rs3743496 | c | 0.0653 | a (test) | 0.0060* |
| Refer to discussion | rs3752158 | g (control) | 0.0207* | c | 0.3359 |
| KCND2 | rs3814463 | t (control) | 0.050* | c | 0.1629 |
| NT_007933 | | | | | |
| KCNQ1 | rs4930127 | g (control) | 0.0309* | a | 0.2581 |
| NT_009237 | | | | | |
| Refer to discussion | rs545118 | a | 0.0754 | t (control) | 0.035 |
| Refer to discussion | rs723672 | g | 0.6552 | t (test) | 0.0168* |
| KCND3 | rs730022 | c (test) | 0.0227* | t | 0.2121 |
| NT_019273 | | | | | |
| Refer to discussion | rs730818 | g (test) | 0.0134* | a | 0.9505 |
| Refer to discussion | rs7578438 | g | 0.1077 | a (test) | 0.0137* |
| Refer to discussion | rs7626962 | g (control) | 0.0094* | t (test) | 0.0094* |
| CACNA1C | rs765125 | t (test) | 0.0335 | c | 0.9409 |
| NT_009759 | | | | | |
| Refer to discussion | rs802351 | t (control) | 0.0310* | c (test) | 0.0151* |

*Fisher's exact test used, as criteria for chi-square test not met
**All patients had at least one allele g so no test was performed

Column 1 indicates the gene and Accession Number for SNPs not previously discussed. Columns 3 and 5 indicate the patient group in parentheses, which had a higher percentage of patients with the allele in question if there was a significant difference. For example, a higher percentage of test patients than control patients had the c allele in the gene SNP rs12276475.

In nine of 15 cases where there was a significant difference between the patient groups in terms of the major allele, the control group had the higher percentage of patients with the allele. In eight of the eleven SNPs in which there was a significant difference between the patient groups in terms of minor allele frequency, the test group had the higher percentage of patients with the minor allele.

Table 19 lists the chromosome position and Build number for each SNP. Table 19 additionally includes the primers used to amplify each SNP and the oligo used for sequencing each SNP. SNPs described elsewhere are indicated.

TABLE 19

SNP
Position/

| | | | |
|---|---|---|---|
| Gene SNP | Build No. | Primers | Oligo |

| | |
|---|---|
| rs12276475 | Refer to discussion |

| | | | |
|---|---|---|---|
| rs1320840 | Chr2: | 5′-aaa atg ttc agc ttg taa ttc ca- | 5′-agg gtc tct acg ctg acg |
| | 26860830 | 3′ (SEQ ID NO. 55) | atc ccc cca aag cta gtg ttt |
| | 111 | 5′-tag aag cta gag agg aaa gtg | agc tct-3′ |
| | | aca a-3′ (SEQ ID NO. 56) | (SEQ ID NO. 57) |
| rs1323860 | Chr11: | 5′-aag agc cat gtg ggc cat-3′ | 5′-aga gcg agt gac gca |
| | 29991812 | (SEQ ID NO. 58) | tac tat ttc agc tca gtt tat |
| | 121 | 5′-aat att ttc aag agt atg ggg ca- | ttt tat ggt-3′ |
| | | 3′ (SEQ ID NO. 59) | (SEQ ID NO. 60) |
| rs1538389 | Chr1: | 5′-ata att ggg agc tgg agt agc t- | 5′-cgt gcc gct cgt gat aga |
| | 112039186 | 3′ (SEQ ID NO. 61) | ata tct tag taa taa tcc caa |
| | 88 | 5′-tca gag gac aca gta tct aag | gca aac-3′ |
| | | gc-3′ (SEQ ID NO. 62) | (SEQ ID NO. 63) |
| rs1808973 | Chr1: | 5′-ttg tgc tgg tgt acc tcg a-3′ | 5′-cgt gcc gct cgt gat aga |
| | 112199876 | (SEQ ID NO. 64) | atg gca gat gat ttg ttg agc |
| | 123 | 5′-aga agg agt aaa ggc agc c-3′ | aga atg-3′ |
| | | (SEQ ID NO. 65) | (SEQ ID NO. 66) |

| | |
|---|---|
| rs1842082 | Refer to the discussion above. |

| | | | |
|---|---|---|---|
| rs1859534 | Chr7: | 5′-agt caa gtc cag tcc aca gta | 5′-agg gtc tct acg ctg acg |
| | 119321505 | ata ta-3′ (SEQ ID NO. 67) | ata tgt gtg ttt gtg ctt ttt |
| | 123 | 5′-aaa ata ctt aag gat ata ctc taa | aga tta-3′ |
| | | agg ca-3′ (SEQ ID NO. 68) | (SEQ ID NO. 69) |

| | |
|---|---|
| rs2072715 | Refer to the discussion |
| rs2238043 | Refer to the discussion |
| rs2239507 | Refer to the discussion |

| | | | |
|---|---|---|---|
| rs2373860 | Chr2: | 5′-att tca act taa gta ttg aat cca | 5′-agc gat ctg cga gac cgt |
| | 40547682 | aag-3′ (SEQ ID NO. 70) | att ttt tct atg gtt ctt atg |
| | 123 | 5′-gtt ttt ttc tct tat ctt tct ttt gtt | gct ata-3′ |
| | | c-3′ (SEQ ID NO. 71) | (SEQ ID NO. 72) |
| rs3739081 | Chr2: | 5′-ata aag aaa gga ggg caa gtg | 5′-cga ctg tag gtg cgt aac |
| | 26867272 | t-3′ (SEQ ID NO. 73) | tca aac agc taa atg caa |
| | 123 | 5′-tcc cac ctg cct ctg tct-3′ | caa tag cag-3′ |
| | | (SEQ ID NO. 74) | (SEQ ID NO. 75) |

| | |
|---|---|
| rs3743496 | Refer to the discussion |
| rs3752158 | Refer to the discussion |

| | | | |
|---|---|---|---|
| rs3814463 | Chr7: | 5′-aat tgg ttg tct tct ggg g-3′ | 5′-agc gat ctg cga gac cgt |
| | 118937980 | (SEQ ID NO. 76) | atg act gga agg caa gac |
| | 120 | 5′-att ttt tgg caa gtt gga ca-3′ | ccg caa agc-3′ |
| | | (SEQ ID NO. 77) | (SEQ ID NO. 78) |
| rs4930127 | Chr 11: | 5′-ctg tgc aga cgc cta agg-3′ | 5′-agc gat ctg cga gac cgt |
| | 2550553 | (SEQ ID NO. 79) | atg tga acc gcg ctg gag |
| | 120 | 5′-tgg gct ata ttg aag ccg-3′ | cgg cgt agg-3′ |
| | | (SEQ ID NO. 80) | (SEQ ID NO. 81) |

| | |
|---|---|
| rs545118 | Refer to the discussion |
| rs723672 | Refer to the discussion |

| | | | |
|---|---|---|---|
| rs730022 | Chr 1: | 5′-gat aac caa gag tta acc ata | 5′-acg cac gtc cac ggt gat |
| | 112227635 | att aca g-3′ (SEQ ID NO. 82) | tta agt aaa aat aaa cta atg |
| | 123 | 5′-taa gta tgc gtg tcc agg aa-3′ | ata ctc-3′ |
| | | (SEQ ID NO. 83) | (SEQ ID NO. 84) |

| | |
|---|---|
| rs730818 | Refer to the discussion |
| rs7578438 | Refer to the discussion |
| rs7626962 | Refer to the discussion |

| | | | |
|---|---|---|---|
| rs765125 | Chr 12: | 5′-tac tgt ggg caa cat ttc ag-3′ | 5′-ggc tat gat tcg caa tgc |
| | 2026468 | (SEQ ID NO. 85) | ttt tgg ggg att tcc ccc aaa |
| | 121 | 5′-atn ctg cac cct ctt cag-3′ | gcc ttc-3′ |
| | | (SEQ ID NO. 86) | (SEQ ID NO. 187) |

| | |
|---|---|
| rs802351 | Refer to the discussion |

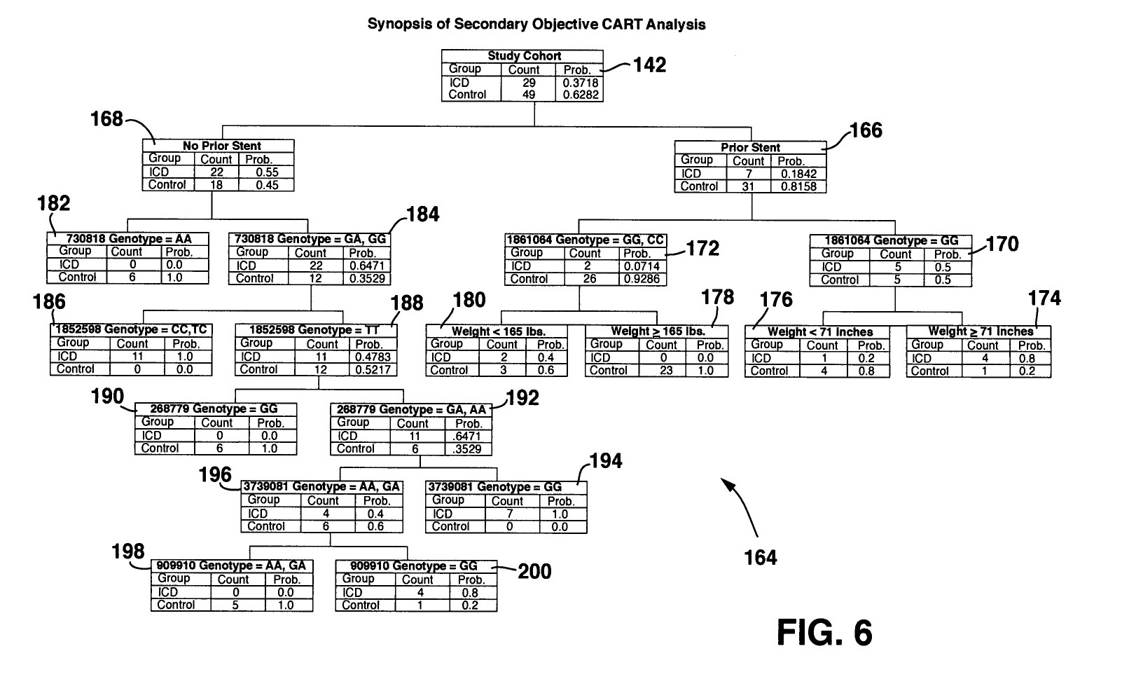

Next, the CART method was used to analyze the relationship between patient genotypes and the likelihood of experiencing a VT/VF episode. Test versus control group was the response variable, and a set of 86 demographic and 162 SNPs were the predictor variables. The resulting tree analysis 164 is shown in FIG. 6.

Tree analysis 164 begins by classifying all patients, represented by group 142, based on the History of Stent variable. Thirty-eight patients had a stent and were placed into group 166, while 40 patients had no stent and were placed into group 168. Of the patients in group 166, only seven, or 18.4% belonged to the test group. Conversely, of the patients in group 168, 22, or 55%, belonged to the test group. The p-value of this clustering using a chi-square test is 0.0007.

Group 166 was further partitioned by SNP rs1861064. Ten patients having the genotype c/g were placed into group 170, while 28 patients having genotypes g/g or c/c were placed into group 172.

Of the patients in group 170, five, or 50%, were test patients. Group 170 was further partitioned based on Patient Height. Five patients from group 170 were greater than or equal to 71 inches and placed into group 174. Four, or 80%, of these patients were test patients and are at risk of VT/VF. The remaining five patients from group 170 were less than 71 inches and placed into group 176. Four, or 80%, of these patients were control patients and, therefore, not at risk of VT/VF.

Patients in group 172 were further partitioned based on Patient Weight. Twenty-three patients from group 172 weighed greater than or equal to 165 lbs. and placed into group 178. All 26 patients were from the control group, and therefore, patients in this group are deemed to not be at significant risk of experiencing VT/VF.

The remaining five patients weighed less than 165 lbs. and placed into group 180. Three patients, or 60%, were control patients, and no further assessment could be made for these patients.

Patients in group 168 were further partitioned based on SNP rs730818. Six patients having the genotype a/a were placed into group 182. All six patients were from the control group. Therefore, patients in group 182 are not at significant risk of VT/VF.

Thirty-four patients having the genotype g/a or g/g were placed into group 184. Group 184 was further partitioned based on SNP rs1852598. Eleven patients from group 184 having the genotype c/c or t/c were placed into group 186. All 11 patients were from the test group. Therefore, patients in group 184 are at significant risk of VT/VF.

The remaining 23 patients from group 184 having the genotype t/t were placed into group 188. Patients in group 188 were further partitioned based on SNP rs268779. Six patients having genotype g/g were placed into group 190. All six patients were from the control group, and therefore, patients in group 190 are not at significant risk of VT/VF.

The remaining 17 patients from group 188 were placed into group 192 and further partitioned based on SNP rs3739081. Seven patients having the genotype g/g were placed into group 194. All seven patients were from the test group and, therefore, deemed to be at significant risk of VT/VF.

Ten patients from group 192 had the genotype a/a or g/a and were placed into group 196. Patients in group 196 were further partitioned based on SNP rs909910. Five patients having genotype a/a or g/a were placed into group 198. All five patients belonged to the control group. Therefore, patients in group 198 are not at significant risk of experiencing fatal VT/VF.

The remaining five patients from group 196 had the genotype g/g and were placed into group 200. Four of the five patients were from the test group. Therefore, patients placed into group 200 are at risk of experiencing fatal VT/VF.

The three analyses indicate that patients who have recently experienced at least one episode of VT/VF differ from those who have not in at least one gene SNP or demographic variable. These SNPs, in turn, can be used as class identifiers for classifying patients. In addition, patients classified as being at low risk earlier in the tree analysis are at lower risk than those classified risk later in the tree analysis. For example, patients in group 182 have a lower risk than patients in group 198. Table 20 summarizes information regarding the SNPs utilized in tree analysis 164.

TABLE 20

SNP
Position/

| | | | |
|---|---|---|---|
| SNP/Gene | Build No. | Primers | Oligo |

| | | | |
|---|---|---|---|
| rs1861064 | Chr7: | 5′-tgt tgt gta tag ctt att atg | 5′-gcg gta ggt tcc cga cat |
| KCND2 | 119132866 | aaa ctg a-3′ | att ata aaa ttt gca gtt tgt tta |
| | | (SEQ ID NO. 88) | ctc-3′ (SEQ ID NO. 90) |
| | 120 | 5′-tta ggc aaa aat gct acc aat | |
| | | c-3′ (SEQ ID NO. 89) | |
| rs268779 | Chr1: | 5′-agt ggg ctc aac ttt tac tgt t- | 5′-gga tgg cgt tcc gtc cta |
| RYR2 | 233736784 | 3′ (SEQ ID NO. 91) | tta atc ttt aat aag aga ggc |
| | 121 | 5′-gaa aga ttc ctg tca ggg c-3′ | agt tac-3′ (SEQ ID NO. 93) |
| | | (SEQ ID NO. 92) | |
| rs909910 | Chr16: | 5′-agc agg tga gtg tcc ttt g-3′ | 5′-acg cac gtc cac ggt gat |
| CACNA1H | 1138939 | (SEQ ID NO. 94) | ttg cca ccc agt cag cag gta |
| | 86 | 5′-tgg atc cca aaa ttc ctt g-3′ | ttt att-3′ (SEQ ID NO. 114) |
| | | (SEQ ID NO. 95) | |

| | |
|---|---|
| rs730818 | Refer to the discussion |
| rs1852598 | Refer to the discussion |
| rs3739081 | Refer to the discussion |

A third genetic study examining 451 SNPs was performed with an additional 12 patients for a total of 90 patient samples included in the study. Here, the control group consists of 52 patients, and the test group consists of 38 patients. The analysis was carried out as previously described, but the results were used to stratify patient risk of experiencing fatal VT/VF or SCD. This stratification scheme can be subsequently used as a class identifier in a classification algorithm.

rs1008832 is located at position chr12:2483782, Build 120, within the CACNA1C gene (Accession No. NT_{—}009759), which codes for the calcium channel, voltage-dependent, L type, alpha 1C subunit protein. This protein is involved in voltage-sensitive calcium channels that mediate the entry of calcium ions into excitable cells and play an important role in excitation-contraction coupling in the heart. The SNP and its flanking regions were amplified using primers 5′-tgc aca tga aca aag ccc-3′ (SEQ ID NO. 96) and 5′-aaa gtt agg aaa gaa gaa gca gaa t-3′ (SEQ ID NO. 97). The SNP was sequenced using the oligo 5′-aga gcg agt gac gca tac taa ggc agg cag cag gtg tga gca gat-3′ (SEQ ID NO. 98). Table 21 shows the statistical breakdown of the genotypes for this SNP.

TABLE 21

| Count | | | |
|---|---|---|---|
| Total (%) | | | |
| Column (%) | | | |
| Row (%) | Control | Test | Total |
| t/t | 28 | 11 | 39 |
| | 31.1% | 12.2% | 43.3% |
| | 53.9% | 29.0% | |
| | 71.8% | 28.2% | |
| t/c | 19 | 20 | 39 |
| | 21.1% | 22.2% | 43.3% |
| | 36.5% | 52.6% | |
| | 48.7% | 51.3% | |
| c/c | 5 | 7 | 12 |
| | 5.6% | 7.8% | 13.3% |
| | 9.6% | 18.4% | |
| | 41.7% | 58.3% | |
| Total | 52 | 38 | 90 |
| | 57.8% | 42.2% | |

The first (top) value in each cell is the number, or count, of patients placed in that set. The second value is the percentage of the total number of patients placed in the set. The third value is the percentage of patients from either the control or test group (depending on the column) placed in the set. The fourth value is the percentage of control or test patients (depending on the column) having a specific genotype from the total number of patients having that specific genotype. The bottom right cell is the total number of patients utilized for the SNP analysis. Here, 90 patients were used, however, in subsequent analysis the total number of patients may be less if a patient's sequence could not be read for a particular SNP. The information in Table 21 was subsequently used to calculate probabilities useful in stratifying patients as to their risk of VT/VF.

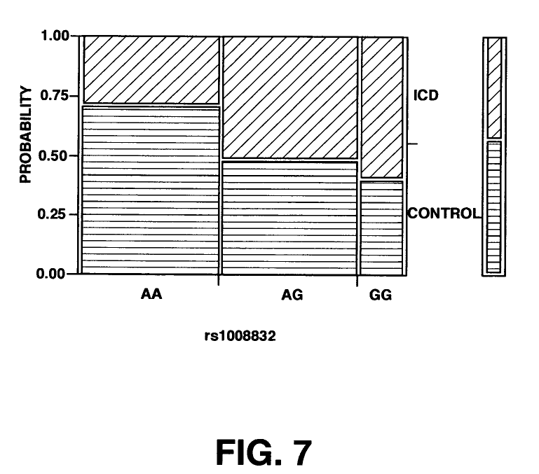

FIG. 7 is a mosaic plot illustrating the resulting risk stratification. The horizontal axis of the graph lists the possible genotypes at this particular SNP. The vertical axis is the probability of experiencing fatal VT/VF.

As shown in the graph, the presence of t at the SNP position indicates increased susceptibility to fatal VT/VF as compared to the presence of c at the SNP position. Specifically, patients with genotype t/t have almost a 75% probability of experiencing fatal VT/VF, while the t/c genotype indicates about a 50% probability, and the c/c genotype indicates about a 40% probability of experiencing fatal VT/VF.

rs2238043 is located at position chr12:2145924, Build 123, within the CACNA1C gene (Accession No. NT_{—}009795). The gene product was described above. The SNP and its flanking regions were amplified using primers 5′-ata cta gac aga gag caa gac ttc aag-3′ (SEQ ID NO. 99) and 5′-tcc cca ttc aaa gtg cct-3′ (SEQ ID NO. 100). The SNP was sequenced using the oligo 5′-aga tag agt cga tgc cag ctg aag tga gat acc taa gga gtg tca-3′ (SEQ ID NO. 101). Table 22 shows the statistical breakdown of the genotypes for this SNP.

TABLE 22

| Count | | | |
|---|---|---|---|
| Total (%) | | | |
| Column (%) | | | |
| Row (%) | Control | Test | Total |
| g/g | 13 | 16 | 29 |
| | 14.4% | 17.8% | 32.2% |
| | 25.0% | 42.1% | |
| | 44.8% | 55.2% | |
| g/a | 29 | 21 | 50 |
| | 32.2% | 23.3% | 55.6% |
| | 55.8% | 55.3% | |
| | 58.0% | 42.0% | |
| a/a | 10 | 1 | 11 |
| | 11.1% | 1.1% | 12.2% |
| | 19.2% | 2.6% | |
| | 90.9% | 9.1% | |
| Total | 52 | 38 | 90 |
| | 57.8% | 42.2% | |

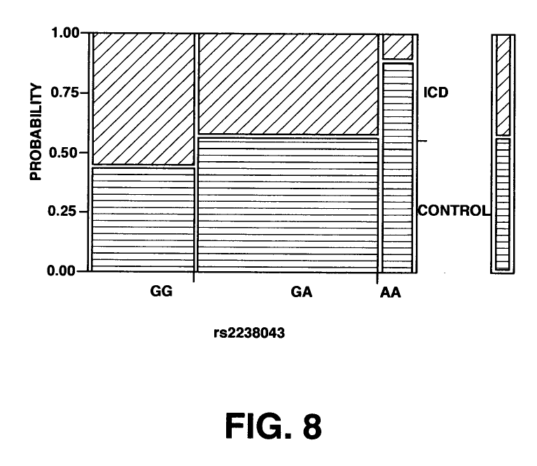

The information in Table 22 was used to calculate probabilities of patient VT/VF. FIG. 8 is a mosaic plot illustrating the resulting risk stratification.

As shown in the plot, the presence of a at the SNP position indicates increased susceptibility to fatal VT/VF as compared to the presence of g at the SNP position. Patients with genotype a/a have about a 90% probability of experiencing fatal VT/VF, while the g/a genotype indicates about a 60% probability, and the g/g genotype indicates about a 45% probability of experiencing fatal VT/VF.

rs198544 was previously described. Table 23 shows the statistical breakdown of the genotypes for this SNP.

TABLE 23

| Count | | | |
|---|---|---|---|
| Total (%) | | | |
| Column (%) | | | |
| Row (%) | Control | Test | Total |
| c/c | 17 | 5 | 22 |
| | 18.9% | 5.6% | 24.4% |
| | 32.7% | 13.2% | |
| | 77.3% | 22.7% | |
| c/g | 27 | 23 | 50 |
| | 30.0% | 25.6% | 55.6% |
| | 51.9% | 60.5% | |
| | 54.0% | 46.0% | |
| g/g | 8 | 10 | 18 |
| | 8.9% | 11.1% | 20.0% |
| | 15.4% | 26.3% | |
| | 44.4% | 55.6% | |
| Total | 52 | 38 | 90 |
| | 57.8% | 42.2% | |

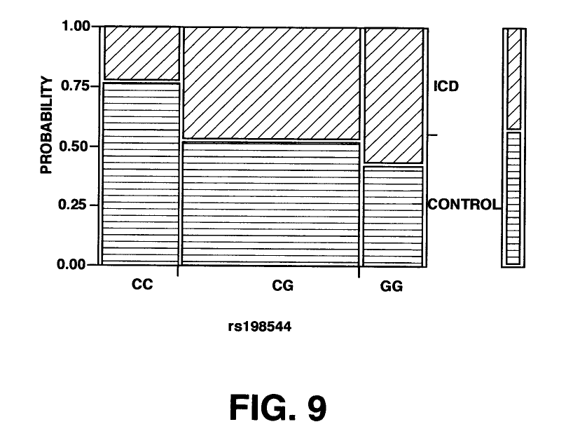

The information in Table 23 was used to calculate probabilities of patient VT/VF. FIG. 9 is a mosaic plot illustrating the resulting risk stratification.

As shown in the plot, the presence of c at the SNP position indicates increased susceptibility to fatal VT/VF as compared to the presence of g at the SNP position. Patients with genotype c/c have just over a 75% probability of experiencing fatal VT/VF, while the c/g genotype indicates about a 50% probability, and the g/g genotype indicates about a 45% probability of experiencing fatal VT/VF.

rs1009531 was previously described. Table 24 shows the statistical breakdown of the genotypes for this SNP.

TABLE 24

| Count | | | |
|---|---|---|---|
| Total (%) | | | |
| Column (%) | | | |
| Row (%) | Control | Test | Total |
| t/t | 2 | 4 | 6 |
| | 2.3% | 4.6% | 6.8% |
| | 3.9% | 10.8% | |
| | 33.3% | 66.7% | |
| t/c | 21 | 20 | 41 |
| | 23.9% | 22.7% | 46.6% |
| | 41.2% | 54.1% | |
| | 51.2% | 48.8% | |
| c/c | 28 | 13 | 41 |
| | 31.8% | 14.8% | 46.6% |
| | 54.9% | 35.1% | |
| | 68.3% | 31.7% | |
| Total | 51 | 37 | 88 |
| | 58.0% | 42.1% | |

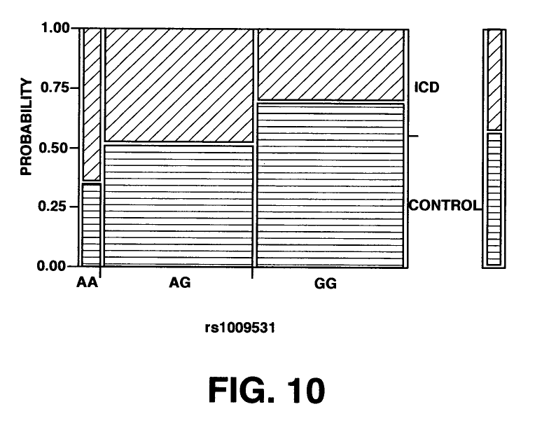

The information in Table 24 was used to calculate probabilities of patient VT/VF. FIG. 10 is a mosaic plot illustrating the resulting risk stratification.

As shown in the plot, the presence of c at the SNP position indicates increased susceptibility to fatal VT/VF as compared to the presence of t at the SNP position. Patients with genotype c/c have about a 70% probability of experiencing fatal VT/VF, while the t/c genotype indicates about a 50% probability, and the t/t genotype indicates about a 35% probability of experiencing fatal VT/VF.

rs2121081 is located at position chr2:155530837, Build 123, within the KCNJ3 gene (Accession No. NT_{—}05403), which codes for the potassium inwardly-rectifying channel, subfamily J, member 3 protein. The protein plays a role in regulating the heartbeat. The SNP and its flanking regions were amplified using primers 5′-aag tga tga aag aaa tga acc ttt-3′ (SEQ ID NO. 102) and 5′-tag agc tgg gat gcg gcc-3′ (SEQ ID NO. 103). The SNP was sequenced using the oligo 5′-aga tag agt cga tgc cag ctg tcg tct gac acc aca gta ctt act-3′ (SEQ ID NO. 104). Table 25 shows the statistical breakdown of the genotypes for this SNP.

TABLE 25

| Count | | | |
|---|---|---|---|
| Total (%) | | | |
| Column (%) | | | |
| Row (%) | Control | Test | Total |
| c/c | 14 | 15 | 29 |
| | 15.6% | 16.7% | 32.2% |
| | 26.9% | 39.5% | |
| | 48.3% | 51.7% | |
| c/g | 24 | 20 | 44 |
| | 26.7% | 22.2% | 48.9% |
| | 46.2% | 52.6% | |
| | 54.6% | 45.5% | |
| g/g | 14 | 3 | 17 |
| | 15.6% | 3.3% | 18.9% |
| | 26.9% | 7.9% | |
| | 82.4% | 17.7% | |
| Total | 52 | 38 | 90 |
| | 57.8% | 42.2% | |

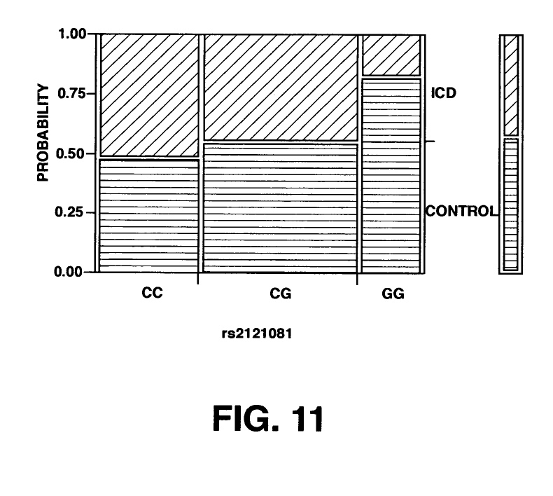

The information in Table 25 was used to calculate probabilities of patient VT/VF. FIG. 11 is a mosaic plot illustrating the resulting risk stratification.

As shown in the plot, the presence of g at the SNP position indicates increased susceptibility to fatal VT/VF as compared to the presence of c at the SNP position. Patients with genotype g/g have about an 85% probability of experiencing fatal VT/VF, while the c/g genotype indicates about a 55% probability, and the c/c genotype indicates just under a 50% probability of experiencing fatal VT/VF.

rs1428568 was described previously. Table 26 shows the statistical breakdown of the genotypes for this SNP.

TABLE 26

| Count | | | |
|---|---|---|---|
| Total (%) | | | |
| Column (%) | | | |
| Row (%) | Control | Test | Total |
| t/t | 9 | 12 | 21 |
| | 10.5% | 14.0% | 24.4% |
| | 18.8% | 31.6% | |
| | 42.9% | 57.1% | |
| t/a | 22 | 17 | 39 |
| | 25.6% | 19.8% | 45.4% |
| | 45.8% | 44.7% | |
| | 56.4% | 43.6% | |
| a/a | 17 | 9 | 26 |
| | 19.8% | 10.5% | 30.2% |
| | 35.4% | 23.7% | |
| | 65.4% | 34.6% | |
| Total | 48 | 38 | 86 |
| | 55.8% | 44.2% | |

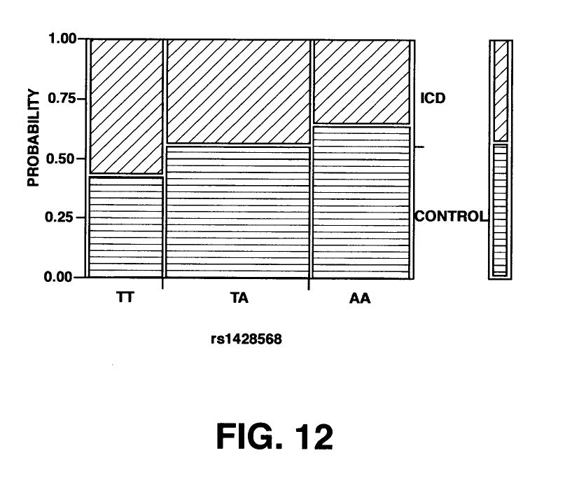

The information in Table 26 was used to calculate probabilities of patient VT/VF. FIG. 12 is a mosaic plot illustrating the resulting risk stratification.

As shown in the plot, the presence of a at the SNP position indicates increased susceptibility to fatal VT/VF as compared to the presence of t at the SNP position. Patients with genotype a/a have about a 65% probability of experiencing fatal VT/VF, while the t/a genotype indicates about a 55% probability, and the t/t genotype indicates about a 40% probability of experiencing fatal VT/VF.

rs918050 was previously described. Table 27 shows the statistical breakdown of the genotypes for this SNP.

TABLE 27

| Count | | | |
|---|---|---|---|
| Total (%) | | | |
| Column (%) | | | |
| Row (%) | Control | Test | Total |
| c/c | 6 | 8 | 14 |
| | 7.2% | 9.6% | 16.9% |
| | 12.5% | 22.9% | |
| | 42.9% | 57.1% | |
| c/t | 16 | 13 | 29 |
| | 19.3% | 15.7% | 34.9% |
| | 33.3% | 37.1% | |
| | 55.2% | 44.8% | |
| t/t | 26 | 14 | 40 |
| | 31.3% | 16.9% | 48.2% |
| | 54.2% | 40.0% | |
| | 65.0% | 35.0% | |
| Total | 48 | 35 | 83 |
| | 57.8% | 42.2% | |

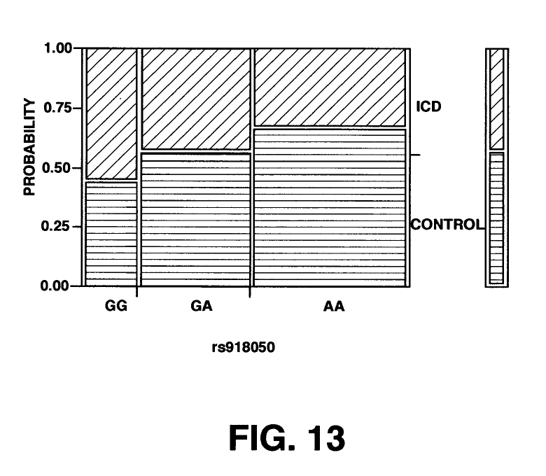

The information in Table 27 was used to calculate probabilities of patient VT/VF. FIG. 13 is a mosaic plot illustrating the resulting risk stratification.

As shown in the plot, the presence of t at the SNP position indicates increased susceptibility to fatal VT/VF as compared to the presence of c at the SNP position. Patients with genotype t/t have about a 65% probability of experiencing fatal VT/VF, while the c/t genotype indicates about a 55% probability, and the c/c genotype indicates about a 40% probability of experiencing fatal VT/VF.

rs1483312 is located at position chr5:45550841, Build 123, within the HCN1 gene (Accession No. NT_{—}006576), which codes for the hyperpolarization activated cyclic nucleotide-gated potassium channel 1 protein. The function of the protein was described above. The SNP and its flanking regions were amplified using primers 5′-tat cct aaa aat cct gct tta att tg-3′ (SEQ ID NO. 105) and 5′-tac atc tag ttg tat agt tct tat ctc taa att atc-3′ (SEQ ID NO. 106). The SNP was sequenced using the oligo 5′-ggc tat gat tcg caa tgc ttg aaa gca tat tac caa taa aaa tta-3′ (SEQ ID NO. 107). Table 28 shows the statistical breakdown of the genotypes for this SNP.

TABLE 28

| Count | | | |
|---|---|---|---|
| Total (%) | | | |
| Column (%) | | | |
| Row (%) | Control | Test | Total |
| t/t | 2 | 6 | 8 |
| | 2.3% | 6.7% | 9.0% |
| | 3.9% | 16.2% | |
| | 25.0% | 75.0% | |
| t/a | 16 | 16 | 32 |
| | 18.0% | 18.0% | 36.0% |
| | 30.8% | 43.2% | |
| | 50.0% | 50.0% | |
| a/a | 34 | 15 | 49 |
| | 38.2% | 16.9% | 55.1% |
| | 65.4% | 40.5% | |
| | 69.4% | 30.6% | |
| Total | 52 | 37 | 89 |
| | 58.4% | 41.6% | |

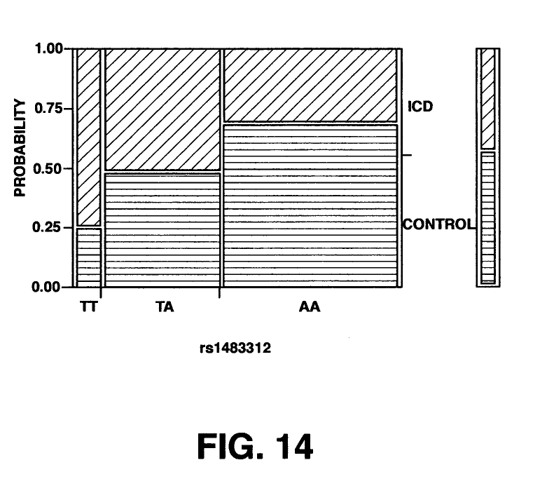

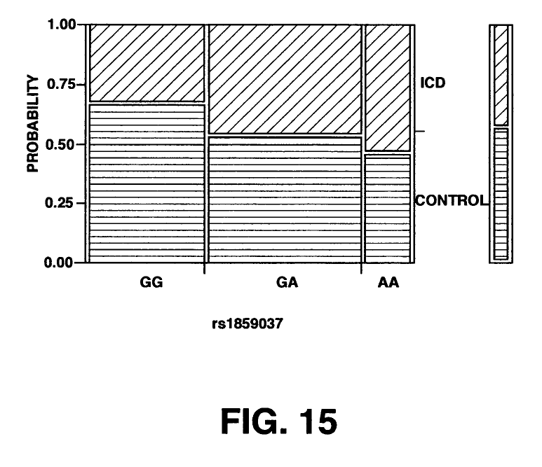

The information in Table 28 was used to calculate probabilities of patient VT/VF. FIG. 14 is a mosaic plot illustrating the resulting risk stratification.

As shown in the plot, the presence of a at the SNP position indicates increased susceptibility to fatal VT/VF as compared to the presence of t at the SNP position. Patients with genotype a/a have about a 70% probability of experiencing fatal VT/VF, while the t/a genotype indicates about a 50% probability, and the t/t genotype indicates about a 25% probability of experiencing fatal VT/VF.

rs1859037 is located at position chr7:90526702, Build 120, within the AKAP9 gene (Accession No. NT_{—}007933), which codes for the A-kinase (PRKA) anchor protein (yotiao) 9. The protein binds to the regulatory subunit of protein kinase A and confines the holoenzyme to discrete locations in a cell. The SNP and its flanking regions were amplified using primers 5′-aat taa tga ttg gta tga caa gtt atg a-3′ (SEQ ID NO. 108) and 5′-tga aag tta gat ttg tgt taa ctt cta tta g-3′ (SEQ ID NO. 109). The SNP was sequenced using the oligo 5′-gac ctg ggt gtc gat acc taa tag gtg cca taa gga aga gtc aga-3′ (SEQ ID NO. 110) Table 29 shows the statistical breakdown of the genotypes for this SNP.

TABLE 29

| Count | | | |
|---|---|---|---|
| Total (%) | | | |
| Column (%) | | | |
| Row (%) | Control | Test | Total |
| g/g | 22 | 11 | 33 |
| | 24.7% | 12.4% | 37.1% |
| | 43.1% | 29.0% | |
| | 66.7% | 33.3% | |
| g/a | 23 | 20 | 43 |
| | 25.8% | 22.5% | 48.3% |
| | 45.1% | 52.6% | |
| | 53.5% | 46.5% | |
| a/a | 6 | 7 | 13 |
| | 6.7% | 7.9% | 14.6% |
| | 11.8% | 18.4% | |
| | 46.2% | 53.9% | |
| Total | 51 | 38 | 89 |
| | 57.3% | 42.7% | |

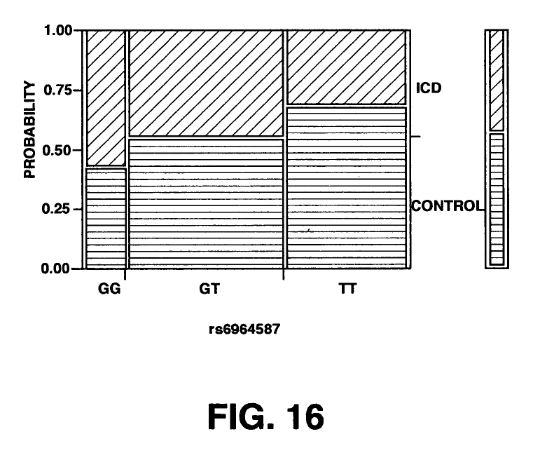

The information in Table 29 was used to calculate probabilities of patient VT/VF. FIG. 16 is a mosaic plot illustrating the resulting risk stratification.

As shown in the plot, the presence of g at the SNP position indicates increased susceptibility to fatal VT/VF as compared to the presence of a at the SNP position. Patients with genotype g/g have about a 65% probability of experiencing fatal VT/VF, while the g/a genotype indicates about a 50% probability, and the a/a genotype indicates about a 45% probability of experiencing fatal VT/VF.

rs6964587 is located at position chr7:90588196, Build 123, within the AKAP9 gene (Accession No. NT_{—}007933), which codes for the A-kinase (PRKA) anchor protein (yotiao) 9. The function of this protein was described above. The SNP and its flanking regions were amplified using primers 5′-gtg caa atg aaa caa gaa tta ata ag-3′ (SEQ ID NO. 111) and 5′-aat atg acc tta aag cat tct cca-3′ (SEQ ID NO. 112). The SNP was sequenced using the oligo 5′-cgt gcc gct cgt gat aga ata aca cat ggc aca gat gga gga aat-3′ (SEQ ID NO. 113). Table 30 shows the statistical breakdown of the genotypes for this SNP.

TABLE 30

| Count | | | |
|---|---|---|---|
| Total (%) | | | |
| Column (%) | | | |
| Row (%) | Control | Test | Total |
| g/g | 5 | 7 | 12 |
| | 5.7% | 8.0% | 13.6% |
| | 10.0% | 18.4% | |
| | 41.7% | 58.3% | |
| g/t | 23 | 20 | 43 |
| | 26.1% | 22.7% | 48.9% |
| | 46.0% | 52.6% | |
| | 53.5% | 46.5% | |
| t/t | 22 | 11 | 33 |
| | 25.0% | 12.5% | 37.5% |
| | 44.0% | 29.0% | |
| | 66.7% | 33.3% | |
| Total | 50 | 38 | 88 |
| | 56.8% | 43.2& | |

The information in Table 30 was used to calculate probabilities of patient VT/VF. FIG. 16 is a mosaic plot illustrating the resulting risk stratification.

As shown in the plot, the presence of t at the SNP position indicates increased susceptibility to fatal VT/VF as compared to the presence of g at the SNP position. Patients with genotype t/t have about a 65% probability of experiencing fatal VT/VF, while the g/t genotype indicates about a 55% probability, and the g/g genotype indicates about a 40% probability of experiencing fatal VT/VF.

Each of these SNP tests can be used individually as class identifiers, or two or more SNP tests may be combined to improve the predictive power.

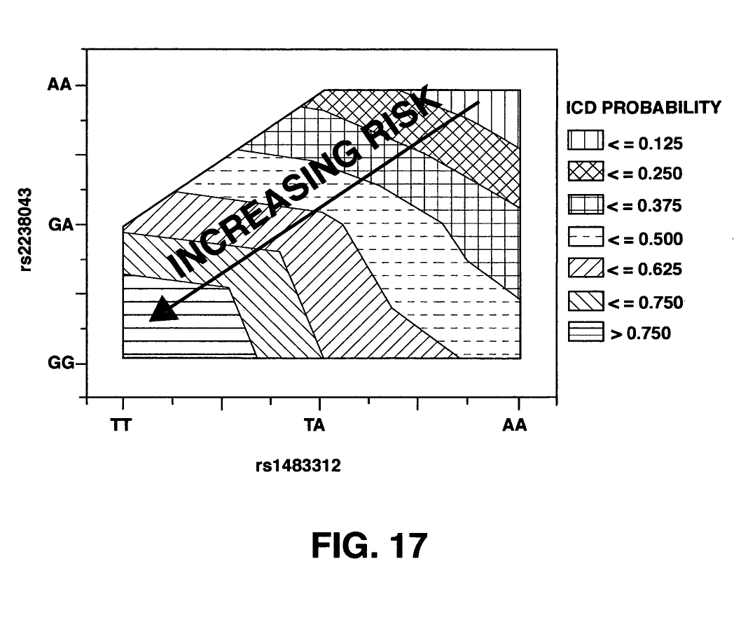

FIG. 17 is a contour plot showing the probability of experiencing VT/VF as a function of the allele specific inheritance pattern of SNPs rs2238043 and rs1483312. The horizontal axis is the possible genotypes of rs1483312, and the vertical axis is the possible genotypes of rs2238043. Matrixes were formed where the points of intersection are the points of interest. The box next to the plot identifies the probabilities that correspond to the intersection points.

The genotype combinations further stratify the patient probabilities to classify patients. For example, a patient having a genotype profile of t/t-g/g has a greater than 75% probability of experiencing VT/VF, while a patient having a genotype profile of t/a-g/a has a less than 75% probability. A patient having a genotype profile of a/a-g/a has a less than 50% probability of experiencing VT/VF, while a patient having a genotype profile of a/a-a/a has a less than 12.5% probability.

A kit that utilizes the present invention includes reagents to extract DNA from biological samples and to subsequently sequence the appropriate SNP or SNPs. The kit would also include a means to convert the determination of SCD-associated SNPs in an individual's genome to susceptibility to SCD. Preferably, the means is a computer algorithm or an algebraic equation, or alternatively, a chart or table may be used to manually look-up the risk.

The above-identified SNPs may also be used in further research to identify and understand the function of factors involved in SCD and to discover new drugs for treatment. It is known that these SNPs may be involved in the action potential, but it is presently unknown how that relates to arrhythmia. In addition, genetic modification of homologous SNPs identified in animals may be made to generate animals with increased probabilities of experiencing SCD. This would create a highly sought animal model for heart disease.

Thus, the present invention provides a means of increasing the sensitivity for identifying individuals with increased susceptibility to SCD due to ventricular arrhythmia. The test method results in gray-level scoring rather than a positive/negative test. In turn, the predictive power of identifying individuals that would benefit from an IMD is also increased.

For example, recent evidence shows that VT/VF is treatable by administration of clonidine or vagal nerve stimulation, as well as through defibrillation by an implantable cardioverter defibrillator (ICD). Thus, SNPs may be used to identify patients that would benefit from these treatments and/or benefit from IMDs such as a drug pump to deliver intrathecal clonidine, a vagal nerve stimulator, or an ICD.

Although the present invention has been described with reference to preferred embodiments, workers skilled in the art will recognize that changes may be made in form and detail without departing from the spirit and scope of the invention.

1. A method of classifying an individual for susceptibility to sudden cardiac death (SCD) comprising:
determining occurrence of at least one SCD-associated polymorphism in a genome of the individual, the SCD-associated polymorphism occurring in one or more genes whose products are involved in cardiac action potential; and
classifying the individual based on determination of the at least one SCD-associated polymorphism.

2. The method of claim 1 wherein the one or more genes are selected from a group consisting of KCND3, SLC8A1, CACNA1G, KCNA5, SCNA5, HCN4, CACNA1I, KCNA4, CACNA1C, HCN2, RYR2, GJA7, KCNJ3, KCND2, KCNK4, KCNQ1, HCN1, and CACNA1H.

3. The method of claim 1 wherein determining occurrence of the at least one SCD-associated polymorphism further comprises:
identifying one or more nucleotides selected from a group of single nucleotide polymorphisms consisting of rs1009531, rs1428568, rs918050, rs198544, rs2239507, rs7626962, rs3743496, rs723672, rs2072715, rs12276475, rs1544503, rs3752158, rs1023214, rs730818, rs1842082, rs7578438, rs545118, rs802351, rs1320840, rs1323860, rs1538389, rs1808973, rs1859534, rs2238043, rs2373860, rs3739081, rs3814463, rs4930127, rs730022, rs765125, rs1861064, rs1852598, rs268779, rs3739081, rs909910, rs1008832, rs2238043, rs2121081, rs1483312, rs1859037, and rs6964587.

4. The method of claim 1 and further comprising:
determining occurrence of at least one class identifier that classifies the individual as having increased susceptibility to SCD;
combining results of occurrences of the SCD-associated polymorphism and the class identifier; and
wherein classifying the individual is based on determination of the combined results.

5. A method of classifying an individual for susceptibility to sudden cardiac death (SCD) comprising:
determining a genotype of the individual for at least one SCD-associated single nucleotide polymorphism (SNP); and
classifying the individual based on determination of the genotype.

6. The method of claim 5 wherein the SCD-associated SNP is selected from a group consisting of rs1009531, rs1428568, rs918050, rs198544, rs2239507, rs7626962, rs3743496, rs723672, rs2072715, rs12276475, rs1544503, rs3752158, rs1023214, rs730818, rs1842082, rs7578438, rs545118, rs802351, rs1320840, rs1323860, rs1538389, rs1808973, rs1859534, rs2238043, rs2373860, rs3739081, rs3814463, rs4930127, rs730022, rs765125, rs1861064, rs1852598, rs268779, rs3739081, rs909910, rs1008832, rs2238043, rs2121081, rs1483312, rs1859037, and rs6964587.

7. The method of claim 5 wherein the susceptibility is stratified based on determination of at least one SCD-associated SNP selected from a group consisting of rs1008832, rs2238043, rs198544, rs1009531, rs2121081, rs1428568, rs918050, rs1483312, rs1859037, and rs6964587.

8. The method of claim 5 wherein the classifying is achieved with an algorithm comprising determination of at least one SCD-associated SNP selected from a group consisting of rs1861064, rs730818, rs1852598, rs268779, rs3739081, and rs909910.

9. A kit for determining relative susceptibility of an individual to sudden cardiac death (SCD), the kit comprising:
reagents for determining, in the individual's genome, at least one SCD-associated polymorphism in one or more genes whose products are involved in cardiac action potential; and
means for converting determination of the at least one SCD-associated polymorphism in the individual's genome to susceptibility to SCD.

10. The kit of claim 9 wherein the SCD-associated polymorphisms are selected from a group consisting of rs1009531, rs1428568, rs918050, rs198544, rs2239507, rs7626962, rs723672, rs2072715, rs12276475, rs1544503, rs3752158, rs1023214, rs730818, rs1842082, rs7578438, rs545118, rs802351, rs1320840, rs1323860, rs1538389, rs1808973, rs1859534, rs2238043, rs2373860, rs3739081, rs3743496, rs3814463, rs4930127, rs730022, rs765125, rs1861064, rs1852598, rs268779, rs3739081, rs909910, rs1008832, rs2238043, rs2121081, rs1483312, rs1859037, and rs6964587.

11. A method of identifying individuals that would benefit from implantation of an implantable medical device, the method comprising:
collecting DNA-containing samples from individuals; and
identifying at least one nucleotide of one or more single nucleotide polymorphisms associated with sudden cardiac death in genomes of the individuals, the single nucleotide polymorphisms selected from a group consisting of rs1009531, rs1428568, rs918050, rs198544, rs2239507, rs7626962, rs3743496, rs723672, rs2072715, rs12276475, rs1544503, rs3752158, rs1023214, rs730818, rs1842082, rs7578438, rs545118, rs802351, rs1320840, rs1323860, rs1538389, rs1808973, rs1859534, rs2238043, rs2373860, rs3739081, rs3814463, rs4930127, rs730022, rs765125, rs1861064, rs1852598, rs268779, rs3739081, rs909910, rs1008832, rs2238043, rs2121081, rs1483312, rs1859037, and rs6964587.

12. A method of optimizing drug therapy for individuals at risk for sudden cardiac death, the method comprising:
collecting DNA-containing samples from individuals; and
identifying at least one nucleotide of one or more single nucleotide polymorphisms associated with sudden cardiac death in genomes of the individuals, the single nucleotide polymorphisms selected from a group consisting of rs1009531, rs1428568, rs918050, rs198544, rs2239507, rs7626962, rs3743496, rs723672, rs2072715, rs12276475, rs1544503, rs3752158, rs1023214, rs730818, rs1842082, rs7578438, rs545118, rs802351, rs1320840, rs1323860, rs1538389, rs1808973, rs1859534, rs2238043, rs2373860, rs3739081, rs3814463, rs4930127, rs730022, rs765125, rs1861064, rs1852598, rs268779, rs3739081, rs909910, rs1008832, rs2238043, rs2121081, rs1483312, rs1859037, and rs6964587.

13. A method of creating an animal model for sudden cardiac death, the method comprising:
modifying a genome of an animal such that the genome contains at least one single nucleotide polymorphism associated with sudden cardiac death, the single nucleotide polymorphism being selected from and homologous to a group consisting of rs1009531, rs1428568, rs918050, rs198544, rs2239507, rs7626962, rs3743496, rs723672, rs2072715, rs12276475, rs1544503, rs3752158, rs1023214, rs730818, rs1842082, rs7578438, rs545118, rs802351, rs1320840, rs1323860, rs1538389, rs1808973, rs1859534, rs2238043, rs2373860, rs3739081, rs3814463, rs4930127, rs730022, rs765125, rs1861064, rs1852598, rs268779, rs3739081, rs909910, rs1008832, rs2238043, rs2121081, rs1483312, rs1859037, and rs6964587.


SEQUENCE LISTING (114 sequences)

SEQ ID NO: 1 - DNA, 24 bp, Homo sapiens
tagacggaagtaaaggttagatcc

SEQ ID NO: 2 - DNA, 18 bp, Homo sapiens
tgtgtttggtgtgggcag

SEQ ID NO: 3 - DNA, 45 bp, Homo sapiens
gtgattctgtacgtgtcgccttttgcctttcctcacagagcttgg

SEQ ID NO: 4 - DNA, 23 bp, Homo sapiens
actctaaaaaatcatgtgccagc

SEQ ID NO: 5 - DNA, 18 bp, Homo sapiens
ttgtggaactggcactgg

SEQ ID NO: 6 - DNA, 45 bp, Homo sapiens
cgactgtaggtgcgtaactctccagctaatgtttgccctcttctc

SEQ ID NO: 7 - DNA, 19 bp, Homo sapiens
cctgcaaagctttcccgta

SEQ ID NO: 8 - DNA, 27 bp, Homo sapiens
ctagaacatgagcaaatacttaattaa

SEQ ID NO: 9 - DNA, 45 bp, Homo sapiens
agatagagtcgatgccagctatggtacaattaagtttaacttaca

SEQ ID NO: 10 - DNA, 20 bp, Homo sapiens
cctggcactaggtgtaaggc

SEQ ID NO: 11 - DNA, 18 bp, Homo sapiens
gaggctggtggtggaaga

SEQ ID NO: 12 - DNA, 45 bp, Homo sapiens
cgtgccgctcgtgatagaatgcagacgtccacagctgcagtcccc

SEQ ID NO: 13 - DNA, 23 bp, Homo sapiens
agtcaggatcaggtatttttcct

SEQ ID NO: 14 - DNA, 19 bp, Homo sapiens
agaacccaggtgaaccaat

SEQ ID NO: 15 - DNA, 45 bp, Homo sapiens
agatagagtcgatgccagcttcatgggtctctgacctcactgtct

SEQ ID NO: 16 - DNA, 18 bp, Homo sapiens
cctccggattccaggacc

SEQ ID NO: 17 - DNA, 18 bp, Homo sapiens
ttccgctttccactgctg

SEQ ID NO: 18 - DNA, 45 bp, Homo sapiens
ggatggcgttccgtcctattagatgcactggcctcggcctcagag

SEQ ID NO: 19 - DNA, 25 bp, Homo sapiens
attgtgttcatttagagaaacagct

SEQ ID NO: 20 - DNA, 18 bp, Homo sapiens
cttctgaggctccccagg

SEQ ID NO: 21 - DNA, 45 bp, Homo sapiens
agggtctctacgctgacgatctgtaacttggagctccactctgcc

SEQ ID NO: 22 - DNA, 22 bp, Homo sapiens
tcagtaggaaatgaaggctttt

SEQ ID NO: 23 - DNA, 19 bp, Homo sapiens
atttcagggaacgaatgga

SEQ ID NO: 24 - DNA, 45 bp, Homo sapiens
gcggtaggttcccgacatattcttcaagcagcgggagggggtggc

SEQ ID NO: 25 - DNA, 24 bp, Homo sapiens
acaaactcaaaggaaaaccataca

SEQ ID NO: 26 - DNA, 20 bp, Homo sapiens
atctcgtcatggcactgagt

SEQ ID NO: 27 - DNA, 45 bp, Homo sapiens
gtgattctgtacgtgtcgcctgggttgttgaatgatacttcagca

SEQ ID NO: 28 - DNA, 21 bp, Homo sapiens
aataggatgcacttgcttgac

SEQ ID NO: 29 - DNA, 21 bp, Homo sapiens
atgaggaagagtcccttcacc

SEQ ID NO: 30 - DNA, 45 bp, Homo sapiens
agggtctctacgctgacgattgatgttcattgatggggacaggca

SEQ ID NO: 31 - DNA, 23 bp, Homo sapiens
tatctgtcacttctacaaccgct

SEQ ID NO: 32 - DNA, 22 bp, Homo sapiens
aattccaaggaggaggaataca

SEQ ID NO: 33 - DNA, 45 bp, Homo sapiens
gcggtaggttcccgacatatatcgggccactgaacaaaacggcaa

SEQ ID NO: 34 - DNA, 18 bp, Homo sapiens
atgcacagcatgtggctc

SEQ ID NO: 35 - DNA, 18 bp, Homo sapiens
tgagcacctgcccaccac

SEQ ID NO: 36 - DNA, 45 bp, Homo sapiens
agggtctctacgctgacgattgcagaaccactcgtggagtgaact

SEQ ID NO: 37 - DNA, 24 bp, Homo sapiens
agtaaagcattatggaggcataaa

SEQ ID NO: 38 - DNA, 28 bp, Homo sapiens
gcatctaagttctcctaaattttttatt

SEQ ID NO: 39 - DNA, 45 bp, Homo sapiens
ggctatgattcgcaatgcttgtttggattatgacatcattctata

SEQ ID NO: 40 - DNA, 20 bp, Homo sapiens
tttttctttcagaagcccct

SEQ ID NO: 41 - DNA, 20 bp, Homo sapiens
acatgacatggtgacaagca

SEQ ID NO: 42 - DNA, 45 bp, Homo sapiens
cgtgccgctcgtgatagaattctttgtcaattgactttttctccc

SEQ ID NO: 43 - DNA, 24 bp, Homo sapiens
aagaaaaaatacaaagacagtggc

SEQ ID NO: 44 - DNA, 21 bp, Homo sapiens
tgtcaaaacctttggttcaaa

SEQ ID NO: 45 - DNA, 45 bp, Homo sapiens
agggtctctacgctgacgatttgttaagcctccttcccgttattc

SEQ ID NO: 46 - DNA, 26 bp, Homo sapiens
catttttacagacaagaaaatttagg

SEQ ID NO: 47 - DNA, 20 bp, Homo sapiens
ataggtttttctctccagcc

SEQ ID NO: 48 - DNA, 45 bp, Homo sapiens
acgcacgtccacggtgatttcttggctgcagaacctctggctaag

SEQ ID NO: 49 - DNA, 25 bp, Homo sapiens
cataaatcttaacttttagcgatcg

SEQ ID NO: 50 - DNA, 29 bp, Homo sapiens
aggatcgttttctagatacaaatgtataa

SEQ ID NO: 51 - DNA, 45 bp, Homo sapiens
agcgatctgcgagaccgtatgatatggtctagatcaacaataatt

SEQ ID NO: 52 - DNA, 29 bp, Homo sapiens
aaaactataagtattttcttgtgaaggtg

SEQ ID NO: 53 - DNA, 19 bp, Homo sapiens
aacatttgccaatgcaatg

SEQ ID NO: 54 - DNA, 45 bp, Homo sapiens
ggatggcgttccgtcctattagcagcatttaaataaatgccctct

SEQ ID NO: 55 - DNA, 23 bp, Homo sapiens
aaaatgttcagcttgtaattcca

SEQ ID NO: 56 - DNA, 25 bp, Homo sapiens
tagaagctagagaggaaagtgacaa

SEQ ID NO: 57 - DNA, 45 bp, Homo sapiens
agggtctctacgctgacgatccccccaaagctagtgtttagctct

SEQ ID NO: 58 - DNA, 18 bp, Homo sapiens
aagagccatgtgggccat

SEQ ID NO: 59 - DNA, 23 bp, Homo sapiens
aatattttcaagagtatggggca

SEQ ID NO: 60 - DNA, 45 bp, Homo sapiens
agagcgagtgacgcatactatttcagctcagtttatttttatggt

SEQ ID NO: 61 - DNA, 22 bp, Homo sapiens
ataattgggagctggagtagct

SEQ ID NO: 62 - DNA, 23 bp, Homo sapiens
tcagaggacacagtatctaaggc

SEQ ID NO: 63 - DNA, 45 bp, Homo sapiens
cgtgccgctcgtgatagaatatcttagtaataatcccaagcaaac

SEQ ID NO: 64 - DNA, 19 bp, Homo sapiens
ttgtgctggtgtacctcga

SEQ ID NO: 65 - DNA, 19 bp, Homo sapiens
agaaggagtaaaggcagcc

SEQ ID NO: 66 - DNA, 45 bp, Homo sapiens
cgtgccgctcgtgatagaatggcagatgatttgttgagcagaatg

SEQ ID NO: 67 - DNA, 26 bp, Homo sapiens
agtcaagtccagtccacagtaatata

SEQ ID NO: 68 - DNA, 29 bp, Homo sapiens
aaaatacttaaggatatactctaaaggca

SEQ ID NO: 69 - DNA, 45 bp, Homo sapiens
agggtctctacgctgacgatatgtgtgtttgtgctttttagatta

SEQ ID NO: 70 - DNA, 27 bp, Homo sapiens
atttcaacttaagtattgaatccaaag

SEQ ID NO: 71 - DNA, 28 bp, Homo sapiens
gtttttttctcttatctttcttttgttc

SEQ ID NO: 72 - DNA, 45 bp, Homo sapiens
agcgatctgcgagaccgtattttttctatggttcttatggctata

SEQ ID NO: 73 - DNA, 22 bp, Homo sapiens
ataaagaaaggagggcaagtgt

SEQ ID NO: 74 - DNA, 18 bp, Homo sapiens
tcccacctgcctctgtct

SEQ ID NO: 75 - DNA, 45 bp, Homo sapiens
cgactgtaggtgcgtaactcaaacagctaaatgcaacaatagcag

SEQ ID NO: 76 - DNA, 19 bp, Homo sapiens
aattggttgtcttctgggg

SEQ ID NO: 77 - DNA, 20 bp, Homo sapiens
attttttggcaagttggaca

SEQ ID NO: 78 - DNA, 45 bp, Homo sapiens
agcgatctgcgagaccgtatgactggaaggcaagacccgcaaagc

SEQ ID NO: 79 - DNA, 18 bp, Homo sapiens
ctgtgcagacgcctaagg

SEQ ID NO: 80 - DNA, 18 bp, Homo sapiens
tgggctatattgaagccg

SEQ ID NO: 81 - DNA, 45 bp, Homo sapiens
agcgatctgcgagaccgtatgtgaaccgcgctggagcggcgtagg

SEQ ID NO: 82 - DNA, 28 bp, Homo sapiens
gataaccaagagttaaccataattacag

SEQ ID NO: 83 - DNA, 20 bp, Homo sapiens
taagtatgcgtgtccaggaa

SEQ ID NO: 84 - DNA, 45 bp, Homo sapiens
acgcacgtccacggtgatttaagtaaaaataaactaatgatactc

SEQ ID NO: 85 - DNA, 20 bp, Homo sapiens
tactgtgggcaacatttcag

SEQ ID NO: 86 - DNA, 18 bp, Homo sapiens
  misc_feature (3)..(3): n is a, c, g, or t
atnctgcaccctcttcag

SEQ ID NO: 87 - DNA, 45 bp, Homo sapiens
ggctatgattcgcaatgcttttgggggatttcccccaaagccttc

SEQ ID NO: 88 - DNA, 28 bp, Homo sapiens
tgttgtgtatagcttattatgaaactga

SEQ ID NO: 89 - DNA, 22 bp, Homo sapiens
ttaggcaaaaatgctaccaatc

SEQ ID NO: 90 - DNA, 45 bp, Homo sapiens
gcggtaggttcccgacatattataaaatttgcagtttgtttactc

SEQ ID NO: 91 - DNA, 22 bp, Homo sapiens
agtgggctcaacttttactgtt

SEQ ID NO: 92 - DNA, 19 bp, Homo sapiens
gaaagattcctgtcagggc

SEQ ID NO: 93 - DNA, 45 bp, Homo sapiens
ggatggcgttccgtcctattaatctttaataagagaggcagttac

SEQ ID NO: 94 - DNA, 19 bp, Homo sapiens
agcaggtgagtgtcctttg

SEQ ID NO: 95 - DNA, 19 bp, Homo sapiens
tggatcccaaaattccttg

SEQ ID NO: 96 - DNA, 18 bp, Homo sapiens
tgcacatgaacaaagccc

SEQ ID NO: 97 - DNA, 25 bp, Homo sapiens
aaagttaggaaagaagaagcagaat

SEQ ID NO: 98 - DNA, 45 bp, Homo sapiens
agagcgagtgacgcatactaaggcaggcagcaggtgtgagcagat

SEQ ID NO: 99 - DNA, 27 bp, Homo sapiens
atactagacagagagcaagacttcaag

SEQ ID NO: 100 - DNA, 18 bp, Homo sapiens
tccccattcaaagtgcct

SEQ ID NO: 101 - DNA, 45 bp, Homo sapiens
agatagagtcgatgccagctgaagtgagatacctaaggagtgtca

SEQ ID NO: 102 - DNA, 24 bp, Homo sapiens
aagtgatgaaagaaatgaaccttt

SEQ ID NO: 103 - DNA, 18 bp, Homo sapiens
tagagctgggatgcggcc

SEQ ID NO: 104 - DNA, 45 bp, Homo sapiens
agatagagtcgatgccagctgtcgtctgacaccacagtacttact

SEQ ID NO: 105 - DNA, 26 bp, Homo sapiens
tatcctaaaaatcctgctttaatttg

SEQ ID NO: 106 - DNA, 36 bp, Homo sapiens
tacatctagttgtatagttcttatctctaaattatc

SEQ ID NO: 107 - DNA, 45 bp, Homo sapiens
ggctatgattcgcaatgcttgaaagcatattaccaataaaaatta

SEQ ID NO: 108 - DNA, 28 bp, Homo sapiens
aattaatgattggtatgacaagttatga

SEQ ID NO: 109 - DNA, 31 bp, Homo sapiens
tgaaagttagatttgtgttaacttctattag

SEQ ID NO: 110 - DNA, 45 bp, Homo sapiens
gacctgggtgtcgatacctaataggtgccataaggaagagtcaga

SEQ ID NO: 111 - DNA, 26 bp, Homo sapiens
gtgcaaatgaaacaagaattaataag

SEQ ID NO: 112 - DNA, 24 bp, Homo sapiens
aatatgaccttaaagcattctcca

SEQ ID NO: 113 - DNA, 45 bp, Homp sapiens
cgtgccgctcgtgatagaataacacatggcacagatggaggaaat

SEQ ID NO: 114 - DNA, 45 bp, Homo sapiens
acgcacgtccacggtgatttgccacccagtcagcaggtatttatt